(19) United States
(12) Patent Application Publication
(10) Pub. No.: US 2026/0248443 A1
(43) Pub. Date: Aug. 27, 2026

(54) MULTIMODAL FUNCTIONAL BRAIN SENSOR

(71) Applicants: Hadasit Medical Research Services and Development Ltd., Jerusalem (IL); A.Y.Y.T. - Technological Applications and Data Update Ltd., Holon (IL)

(72) Inventors: Michal BALBERG, Jerusalem (IL); Mordekhay MEDVEDOVSKY, Beer Sheva (IL); Evgeny TSIZIN-GOLDMAN, Ramat Gan (IL)

(21) Appl. No.: 18/871,028

(22) PCT Filed: Jun. 12, 2023
(86) PCT No.: PCT/IL2023/050606

§ 371 Date: Dec. 2, 2024

Related U.S. Application Data
(60) Provisional application No. 63/351,404, filed on Jun. 12, 2022.

(51) Int. Cl. A61B 5/00; A61B 5/0536; A61B 5/1455; A61B 5/1459

(52) U.S. Cl. CPC A61B 5/4094; A61B 5/0536; A61B 5/14553; A61B 5/1459; A61B 5/4064; A61B 5/6864

(57) ABSTRACT

An apparatus includes an intraosseous appliance formed to have hollow channels open to a proximal extradermal surface of the appliance. Optical emitters and detectors reside within channels of the intraosseous appliance having distal openings in a distal base of the intraosseous appliance, in a lateral surface of the intraosseous appliance, and in an extradermal surface of the intraosseous appliance, so as to establish optical paths between the optical emitters and detectors and, respectively, the dura, bone wall of a bun hole and skin of the subject. Intracerebral and extracerebral electrodes reside within respective channels of the intraosseous appliance having distal openings in the distal base of the intraosseous appliance and in the extradermal surface of the intraosseous appliance, respectively.

### FIELD

The present disclosure relates to articles of manufacture comprising apparatuses for use in brain monitoring, and to methods for their use, and particularly to their application in localization of epileptogenic foci and characterization of neuro-vascular coupling during ictal, preictal and interictal periods.

### BACKGROUND

Epilepsy is a common neurological disease, defined as an enduring predisposition to have epileptic seizures (excessive synchronous neuronal electrical activity in part of the brain's cortex, manifesting in transient changes in the subject's behavior), and affecting about 1% of the population worldwide. Despite the development of multiple drugs over the last decades, about a third of patients continue to have seizures that are resistant to medications. A proportion of them are candidates for invasive treatments, aimed to prevent seizures by destroying the tissue or modulating the electrical activity in the seizures' origin, i.e. the epileptogenic area. Thus, an imperative step in the diagnosis of medications-resistant focal epilepsy is the correct localization and delineation of the epileptogenic area within the patient's brain tissue. Although multiple advanced techniques are used to that end, the epileptic focus is many times not identified correctly, resulting in a relatively low success rate of the non-pharmacological treatments, which range between 50 and 90%. Therefore, there is need for better techniques to accurately localize the epileptic tissue in the brain and ultimately improve the outcomes of treatments.

### SUMMARY

According to embodiments of the presently disclosed subject matter, an intraosseous appliance useful for brain monitoring is configured (formed) to hold at least one optode (i.e. an optical component such as an optical emitter configured to deliver light such as near infrared light or a detector configured to measure light e.g. diffusely reflected near infrared light) so as that said optode can provide direct optical access (unimpeded passage of light without significant scattering, absorption and/or distortion) beyond a scalp of said patient. The intraosseous appliance may provide a stable optical access beyond the scalp of the patient. Optodes may include: optical fibers, optical detectors such as photodiodes or optical emitters such as LED or lasers. Optical fibers offer flexibility in the design, while detectors and emitters can also be inserted inside the appliances, with or without power sources. The intraosseous appliance may also be formed to additionally hold at least one electrode such as a surface electrode configured to record and/or stimulate electrical activity on the scalp surface or a depth electrode configured to record and/or stimulate electrical activity within brain tissue. The intraosseous appliance can be inserted into the skull of patients with epilepsy. The electrodes and/or optical components can be inserted into the brain and/or placed in proximity to the brain, to directly measure the brain vasculature, electrical activity and electrical impedance (EIT—electric impedance tomography). These measurements may enable better localization of ictal and interictal epileptic activities while substantially reducing motion artifacts and contamination from extracerebral factors. Arrays of such appliances are complemented by multi-modal software that can co-register received optical and electrical signals with whole-brain imaging. The application of the presently disclosed subject matter is not limited to epilepsy, rather it can be used in combination with device for intra-cranial pressure measurement and with intracranial insertion through the burr hole of any device (depth electrodes, laser probes, catheters and other) for recording, modulation, ablation, biopsy or substance delivery.

The presently disclosed subject matter provides an intraosseous appliance configured for (optionally reversible) insertion in a burr hole of a patient skull, the intraosseous appliance comprising at least one optode channel extending through the intraosseous appliance, said optode channel being configured to accommodate an optode and to provide direct optical access beyond a scalp of said patient. It is understood that direct optical access is provided when an optode is inserted in said optode channel and said intraosseous appliance is inserted in the patient skull burr hole.

In addition with the above features, the intraosseous appliance of the presently disclosed subject matter can optionally comprise one or more of features (i) to (xvii) below, any technically possible combination or permutation:
(i) the at least one optode channel comprises one or more brain optode channel configured to provide direct optical access to a dura of the patient. The one or more brain optode channel may have respective distal openings in a distal base of the intraosseous appliance. The one or more brain optode channel may form one or more linear channel parallel to a longitudinal axis of the intraosseous appliance.
(ii) the at least one optode channel comprises one or more skull wall optode channel configured to provide direct optical access to a lateral skull wall of the patient burr hole. The one or more skull wall optode channel may have respective distal openings in a lateral surface of the intraosseous appliance. This may enable to avoid a potential blood clot created by bolting the intraosseous appliance in proximity to the patient dura to contaminate optical imaging. The one or more skull wall optode channel may form a helix or a curve. Said helix or curve may have a proximal extradermal opening and a distal opening in a lateral surface of the intraosseous appliance.
(iii) at least one extradermal optode channel extending through an extradermal portion of the intraosseous appliance, said extradermal optode channel being configured to accommodate an extradermal optode and to provide direct optical access to a skin of a scalp portion peripheral to the patient burr hole.
(iv) a depth electrode channel extending through the intraosseous appliance, said depth electrode channel being configured to accommodate a depth electrode and to provide direct access to a dura of the patient. Optionally, the depth electrode channel may be tilted relative to a longitudinal axis of the intraosseous appliance. This facilitates penetration of the depth electrode in the brain. In other embodiments, the depth electrode channel may extend along the longitudinal axis and optionally be centered relative to the intraosseous appliance.
(v) a surface electrode channel being configured to accommodate a surface electrode and to provide access to a scalp surface.
(vi) at least one of the optode channel and the extradermal optode channel is configured for accommodating an optode in the form of an optical fiber or an optical fiber bundle.
(vii) a stabilization structure configured for securing the intraosseous appliance in the cranial burr hole.
(viii) the stabilization structure includes a threading on at least part of a lateral surface of the intraosseous appliance.
(ix) the appliance is formed from one or more non-ferromagnetic materials.
(x) a distal portion of the appliance is formed from a material that is substantially transparent to a least a portion of a 400 nm-2 μ section of an electromagnetic spectrum.
(xi) the distal portion is an integrally formed portion of the intraosseous appliance.
(xii) an extradermal annular flange portion.
(xiii) the extradermal optode channel extends through the extradermal annular flange portion.
(xiv) the at least one optode channel is included in a hollow channel array that includes a plurality of channels having respective proximal openings in a proximal extradermal surface of the intraosseous appliance, the hollow channel array comprising at least one array subset selected from:
   (a) a first array subset comprising a plurality of channels adapted for respective passage therethrough of a first group of optical components including at least one emitter element and at least one detector element, and a first electrode array comprising at least one electrode, the channels of the first array subset having respective distal openings in a distal base of the intraosseous appliance;
   (b) a second array subset comprising a plurality of channels adapted for respective passage therethrough of a second group of optical components including at least one emitter element and at least one detector element, the channels of the second array subset having respective distal openings in a lateral surface of the intraosseous appliance.
(xv) a third array subset comprising a plurality of channels adapted for respective passage therethrough of a third group of optical components including at least one emitter element and at least one detector element, and a second electrode array comprising at least one electrode, the channels of the third array subset having respective distal openings in an extradermal surface of the intraosseous appliance that is not the proximal extradermal surface.
(xvi) the respective proximal and distal openings of the channels of the third array subset are in opposing faces of the flange portion.
(xvii) at least one designated channel for passage therethrough of a respective sensor selected from a thermal sensor, an intercranial pressure sensor, an oxygen tension sensor, and a blood-flow sensor.

The presently disclosed subject matter also provides an apparatus including an intraosseous appliance as described and at least one optode configured to be accommodated in said respective optode channel to provide direct optical access beyond a scalp of said patient. The optode may be configured for enabling fNIRS brain monitoring. This enables preventing contamination of the fNIRS signal by the extracerebral blood flow of the scalp.

In addition with the above features, the apparatus previously disclosed can optionally comprise one or more of features (i) to (xvi) below, in any technically possible combination or permutation:
(i) the at least one optode channel comprises one or more brain optode channel configured to provide direct optical access to a dura of the patient and the apparatus includes a brain optode configured to be accommodated in the brain optode channel. The one or more brain optode channel may have respective distal openings in a distal base of the intraosseous appliance.
(ii) the at least one optode channel comprises one or more skull wall optode channel configured to provide direct optical access to a lateral skull wall of the patient burr hole and the apparatus includes a skull wall optode configured to be accommodated in the skull wall optode channel. The one or more skull wall optode channel may have respective distal openings in a lateral surface of the intraosseous appliance.
(iii) at least one extradermal optode channel extending through an extradermal portion of the intraosseous appliance, said extradermal optode channel being configured to accommodate an extradermal optode and to provide direct optical access to a skin of a scalp portion peripheral to the patient burr hole, and the apparatus includes an extradermal optode configured to be accommodated in the extradermal optode channel.
(iv) a depth electrode channel extending through the intraosseous appliance, said depth electrode channel being configured to accommodate a depth electrode and to provide direct access to a dura of the patient, and the apparatus includes depth electrode configured to be accommodated in the depth electrode channel. Optionally, the depth electrode channel may be tilted relative to a longitudinal axis of the intraosseous appliance. This facilitates penetration of the depth electrode in the brain. In other embodiments, the depth electrode channel may extend along the longitudinal axis and optionally be centered relative to the intraosseous appliance. Optionally, the depth electrode includes one or more intracerebral optical components such as an intracerebral emitter configured to deliver near infrared light and/or an intracerebral detector configured to detect near infrared light within brain tissue. In some embodiments, an emission aperture or a detection aperture of said intracerebral emitter an/or detector may be tilted relative to an axis of the depth electrode channel.
(v) a surface electrode channel being configured to accommodate a surface electrode and to provide access to a scalp surface, and the apparatus includes a surface electrode configured to be accommodated in the surface electrode channel.
(vi) at least one of the optode channel (brain optode channel and/or skull wall optode channel) and the extradermal optode channel is configured for accommodating an optode in the form of an optical fiber or an optical fiber bundle and the optode and/or the extradermal optode is in the form of an optical fiber.
(vii) a stabilization structure configured for securing the intraosseous appliance in the cranial burr hole.
(viii) the stabilization structure includes a threading on at least part of a lateral surface of the intraosseous appliance.
(ix) The intraosseous appliance is formed from one or more non-ferromagnetic materials.
(x) a distal portion of the intraosseous appliance is formed from a material that is substantially transparent to a least a portion of a 400 nm-2 μ section of an electromagnetic spectrum.
(xi) the distal portion is an integrally formed portion of the intraosseous appliance.
(xii) The intraosseous appliance includes an extradermal annular flange portion.
(xiii) the extradermal optode channel extends through the extradermal annular flange portion.
(xiv) the at least one optode channel is included in a hollow channel array that includes a plurality of channels having respective proximal openings in a proximal extradermal surface of the intraosseous appliance, the hollow channel array comprising at least one array subset selected from:
   (a) a first array subset comprising a plurality of channels adapted for respective passage therethrough of a first group of optical components including at least one emitter element and at least one detector element, and a first electrode array comprising at least one electrode, the channels of the first array subset having respective distal openings in a distal base of the intraosseous appliance;
   (b) a second array subset comprising a plurality of channels adapted for respective passage therethrough of a second group of optical components including at least one emitter element and at least one detector element, the channels of the second array subset having respective distal openings in a lateral surface of the intraosseous appliance.
   (c) a third array subset comprising a plurality of channels adapted for respective passage therethrough of a third group of optical components including at least one emitter element and at least one detector element, and a second electrode array comprising at least one electrode, the channels of the third array subset having respective distal openings in an extradermal surface of the intraosseous appliance that is not the proximal extradermal surface;
      and the apparatus includes:
      at least one group of optical components configured for performing fNIRS, each of the optical components comprising one of an emitter and a detector, the at least one group selected from: (i) a first group of optical components adapted to reside at least partly within respective channels having distal openings in a distal base of the intraosseous appliance so as to establish respective optical paths between respective distal ends of the first-group optical components and a dura of the subject, (ii) a second group of optical components adapted to reside at least partly within respective channels having distal openings in a lateral surface of the intraosseous appliance so as to establish respective optical paths between respective distal ends of the second-group optical components and a bone wall of the burr hole, and (iii) a third group of optical components adapted to reside at least partly within respective channels having distal openings in an extradermal surface of the intraosseous appliance that is not the proximal extradermal surface so as to establish respective optical paths between respective distal ends of the third-group optical components and skin of the subject; and
      an electrode array including at least one electrode subarray selected from: (i) a first electrode subarray comprising one or more intracerebral electrodes adapted to reside at least partly within respective channels having distal openings in a distal base of the intraosseous appliance, and (ii) a second electrode subarray comprising one or more extracerebral electrodes adapted to reside at least partly within respective channels having distal openings in the extradermal surface of the intraosseous appliance that is not the proximal extradermal surface. The intracerebral electrode may be configured for performing stereo-electroencephalography. Optionally, the one or more intracerebral electrodes include intracerebral optical components. The extracerebral electrodes may be configured for detecting and imaging electrical impedance change using electrical impedance tomography (EIT), optionally performed within a lower range of two non-overlapping frequency ranges or within an upper range of the two non-overlapping frequency ranges.
(xv) the respective proximal and distal openings of the channels of the third array subset are in opposing faces of the flange portion.
(xvi) the intraosseous appliance includes at least one designated channel for passage therethrough of a respective sensor selected from a thermal sensor, an intercranial pressure sensor, an oxygen tension sensor, and a blood-flow sensor, and the apparatus includes said respective sensor being configured to be accommodated in said designated channel.

The presently disclosed subject matter also provides a system for performing brain monitoring and/or brain mapping comprising one or more apparatus as previously described, said system including at least one emitter and at least one detector and electronic circuitry configured for enabling brain monitoring and/or brain mapping. The electronic circuitry may be in communication with the at least one emitter and at least one detector optionally one or more of the first, second and third groups of optical components, and from one or more of the first and second electrode arrays.

In addition with the above features, the system of the presently disclosed subject matter can optionally comprise one or more of features (i) to (ii) below, in any technically possible combination or permutation:
(i) each apparatus comprises one or more intracerebral optical components and an intracerebral optical component of an apparatus is configured to detect an emission by an intracerebral optical component of another apparatus.
(ii) The intracerebral optical components are arranged on respective intracerebral (depth) electrodes of a respective apparatus.

The presently disclosed subject matter also provides a method of monitoring a human brain using the appliances, apparatuses and systems disclosed herein.

The presently disclosed subject matter also provides an apparatus including (i) an intraosseous appliance configured for reversible insertion in a burr hole of a patient skull, the intraosseous appliance comprising at least one depth electrode channel extending through the intraosseous appliance, said depth electrode channel being configured to accommodate a depth electrode and to provide direct access to a dura of said patient and (ii) a depth electrode configured to record and/or stimulate electrical activity within brain tissue, wherein the depth electrode additionally include at least one of an intracerebral emitter configured to deliver near infrared light or an intracerebral detector configured to detect near infrared light within brain tissue.

The presently disclosed subject also provides a system for performing brain monitoring and/or brain mapping including a plurality of apparatuses as previously disclosed wherein an intracerebral detector of an apparatus is configured to detect an emission by an intracerebral emitter of another apparatus.

In some embodiments, at least one apparatus includes a depth electrode having a plurality of intracerebral emitters and another apparatus includes a depth electrode with a detector configured for detecting signal from at least some of the plurality of intracerebral emitter from said at least one apparatus.

According to embodiments of the presently disclosed subject matter, an apparatus comprises an intraosseous appliance comprising a hollow-channel array that includes a plurality of channels having respective proximal openings in a proximal extradermal surface of the intraosseous appliance, the hollow-channel array comprising at least one array subset selected from: (i) a first array subset comprising a plurality of channels adapted for respective passage therethrough of (A) a first group of optical components including at least one emitter element and at least one detector element, and (B) a first electrode array comprising at least one electrode, the channels of the first array subset having respective distal openings in a distal base of the intraosseous appliance that is opposite the proximal surface, (ii) a second array subset comprising a plurality of channels adapted for respective passage therethrough of a second group of optical components including at least one emitter element and at least one detector element, the channels of the second array subset having respective distal openings in a lateral surface of the intraosseous appliance, and (iii) a third array subset comprising a plurality of channels adapted for respective passage therethrough of (A) a third group of optical components including at least one emitter element and at least one detector element, and (B) a second electrode array comprising at least one electrode, the channels of the third array subset having respective distal openings in an extradermal surface of the intraosseous appliance that is not the proximal extradermal surface.

In some embodiments, the hollow-channel array can comprise at least two of the first, second and third array subsets. In some embodiments, the hollow-channel array can comprise the first, second and third array subsets.

In some embodiments, the intraosseous appliance can include a threading on at least part of the lateral surface of the intraosseous appliance. In some embodiments, the intraosseous appliance can be formed from one or more non-ferromagnetic materials.

In some embodiments, the intraosseous appliance can comprise a distal portion formed from a material that is substantially transparent to a least a portion of a 400 nm-1 μ section of an electromagnetic spectrum. In some embodiments, the intraosseous appliance can comprise a distal portion formed to allow passage therethrough of electromagnetic radiation of at least one wavelength in a 400 nm-1 μ section of an electromagnetic spectrum. In some embodiments, the distal portion can be an integrally formed portion of the intraosseous appliance. In some embodiments, the distal portion can be joined to the distal base of the intraosseous appliance and/or can comprise one or more volumes contiguous to at least some of the respective distal openings of the channels of the first array subset.

In some embodiments, the intraosseous appliance can comprise an extradermal annular flange portion having a maximum outer diameter of the intraosseous appliance. In some such embodiments, the respective proximal and distal openings of the channels of the third array subset can be in opposing faces of the flange section.

In some embodiments, the first array subset can comprise a designated channel adapted for respective passage therethrough of an intracerebral electrode. In some such embodiments, the designated channel can traverse the intraosseous appliance from a respective proximal opening in the proximal extradermal surface to a respective distal opening in the distal base at an angle of at least 5° from a central longitudinal axis of the appliance, or at an angle of at least 10° therefrom, or at an angle of at least 15° therefrom, or at an angle of at least 20° therefrom.

In some embodiments, the hollow-channel array can comprise at least one designated channel for passage therethrough of a respective sensor selected from a thermal sensor, an intercranial pressure sensor, an oxygen tension sensor, and a blood-flow sensor.

In some embodiments, the apparatus can additionally comprise: (a) the first, second and third groups of optical elements, each of the optical components comprising (i) a wire and (ii) one of an emitter and a detector, and/or (b) the electrodes of the first and second electrode arrays. In some such embodiments, the apparatus can additionally comprise at least one sensor selected from a thermal sensor, an intercranial pressure sensor, an oxygen tension sensor, and a blood-flow sensor. In some such embodiments, the apparatus can be provided in an assembled state, such that the first, second and third groups of optical-sensor elements can be resident in respective channels of the first, second and third array subsets. In some embodiments, a kit can comprise the apparatus in said assembled state.

According to embodiments of the presently disclosed subject matter, an apparatus comprises: (a) an intraosseous appliance shaped for insertion in, and removal from, a cranial burr hole, the intraosseous appliance comprising a plurality of channels having respective proximal openings in a proximal extradermal surface of the intraosseous appliance; (b) at least one group of optical components, each of the optical components comprising one of an emitter and a detector, the at least one group selected from: (i) a first group of optical components adapted to reside at least partly within respective channels having distal openings in a distal base of the intraosseous appliance so as to establish respective optical paths between respective distal ends of the first-group optical components and a dura of the subject, (ii) a second group of optical components adapted to reside at least partly within respective channels having distal openings in a lateral surface of the intraosseous appliance so as to establish respective optical paths between respective distal ends of the second-group optical components and a bone wall of the burr hole, and (iii) a third group of optical components adapted to reside at least partly within respective channels having distal openings in an extradermal surface of the intraosseous appliance that is not the proximal extradermal surface so as to establish respective optical paths between respective distal ends of the third-group optical components and skin of the subject; and (c) an electrode array including at least one electrode subarray selected from: (i) a first electrode subarray comprising one or more intracerebral electrodes adapted to reside at least partly within respective channels having distal openings in a distal base of the intraosseous appliance, and (ii) a second electrode subarray comprising one or more extracerebral electrodes adapted to reside at least partly within respective channels having distal openings in the extradermal surface of the intraosseous appliance that is not the proximal extradermal surface.

In some embodiments, the apparatus can comprise at least two groups of optical components selected from the first, second and third groups of optical components. In some embodiments, the apparatus can comprise the first, second and third groups of optical components. In some embodiments, the apparatus can additionally comprise one or more intracerebral optical components. In some embodiments, the apparatus can additionally comprise one or more intracerebral optical components joined to respective intracerebral electrodes.

In some embodiments, at least some of the optical components can be configured for functional near infrared spectroscopy (fNIRS) surveillance of hemodynamic responses. In some embodiments, at least some of the optical components can be configured for fNIRS surveillance of neuronal responses. In some embodiments, at least some of the optical components can be configured for measuring a change a concentration of oxyhemoglobin and/or hemoglobin in the vicinity of the at least some of the optical components. In some embodiments, at least some of the electrodes of the electrode array can be configured for detecting and imaging electrical impedance change using electrical impedance tomography (EIT).

In some embodiments, at least some of the electrodes can be configured to perform EIT in at least two frequency ranges. In some such embodiments, the at least two frequency ranges can be non-overlapping; a first (lower) frequency range can be below an upper threshold of 500 Hz, or 1 kHz, or 1.5 kHz, or 2 kHz, and/or a second (upper) frequency range can be above a lower threshold of 3 kHz, or 2 kHz, or 1 kHz.

In some embodiments, at least some of the electrodes of the electrode array can be configured for monitoring an electric field.

In some embodiments, the intraosseous appliance can include a threading on at least part of the lateral surface of the intraosseous appliance.

In some embodiments, the intraosseous appliance can be formed from one or more non-ferromagnetic materials.

In some embodiments, the intraosseous appliance can comprise a distal portion formed from a material that is substantially transparent to a least a portion of a 400 nm-1 μ section of an electromagnetic spectrum. In some embodiments, the intraosseous appliance can comprise a distal portion formed to allow passage therethrough of a majority of available electromagnetic radiation in at least a portion of a 400 nm-1 μ section of an electromagnetic spectrum.

In some embodiments, the distal portion can be an integrally formed portion of the intraosseous appliance. In some embodiments, the distal portion can be joined to the distal base of the intraosseous appliance and/or can comprise one or more volumes contiguous to at least some of the respective distal openings of the channels of the first array subset.

In some embodiments, the intraosseous appliance can comprise an extradermal annular flange portion having a maximum outer diameter of the intraosseous appliance. In some such embodiments, the annular flange can comprise two opposing surfaces, e.g., a first opposing surface including at least a portion of the extradermal surface that is not the proximal extradermal surface, and/or a second opposing surface including at least a portion of the proximal extradermal surface.

In some embodiments, the apparatus can additionally comprise a thermal sensor arranged to monitor a temperature of the appliance or a tissue.

In some embodiments, the apparatus is provided in an assembled state, e.g., such that the optical components of the at least one group of optical components are at least partly resident in the respective channels. In some embodiments, the apparatus is provided in the assembled state, e.g., such that the optical components of the first, second and third groups of optical components are at least partly resident in the respective channels. In some such embodiments, the respective one or more electrodes of at least one of the first and second electrode subarrays can be at least partly resident in the respective channels. In some embodiments, a method for monitoring a human brain can comprise: (a) providing the apparatus in the assembled state, with the intraosseous appliance arranged partly within a cranial burr hole; (b) receiving, from optical components residing at least partly in the respective channels, information about hemodynamic responses and/or neuronal responses; and (c) further receiving, from at least one electrode residing at least partly in the respective channels, information about electrical impedance and/or an electric field.

In some embodiments, a plurality of apparatuses according to any of the foregoing embodiments can comprise first and second apparatuses including respective intracerebral optical components, and/or an intracerebral optical component of the first apparatus can be configured to detect an emission by an intracerebral optical component of the second apparatus. In some embodiments, a system can comprise such a plurality of apparatuses, and/or electronic circuitry for performing brain mapping using information received from at least one optical component of the at least one group of optical components and/or from at least one electrode of the electrode array.

A method is disclosed, according to embodiments of the presently disclosed subject matter, for monitoring a human brain. The method comprises: (a) inserting, in a cranial burr hole, an intraosseous appliance comprising a plurality of channels having respective proximal openings in a proximal extradermal surface; (b) positioning at least one group of optical components at least partly within a first subset of the channels, each of the optical components comprising one of an emitter and a detector, the positioning including at least one of: (i) positioning a first group of optical components within respective channels having distal openings in a distal base of the intraosseous appliance so as to establish respective optical paths between respective distal ends of the first-group optical components and a dura of the subject, (ii) positioning a second group of optical components adapted to reside at least partly within respective channels having distal openings in a lateral surface of the intraosseous appliance so as to establish respective optical paths between respective distal ends of the second-group optical components and a bone wall of the burr hole, and (iii) positioning a third group of optical components adapted to reside at least partly within respective channels having distal openings in an extradermal surface of the intraosseous appliance that is not the proximal extradermal surface so as to establish respective optical paths between respective distal ends of the third-group optical components and skin of the subject; and (c) further positioning at least one electrode array at least partly within a second subset of the channels, the further positioning including at least one of: (i) positioning a first electrode subarray comprising one or more intracerebral electrodes within respective channels having distal openings in a distal base of the intraosseous appliance, and (ii) positioning a second electrode subarray comprising one or more extracerebral electrodes within respective channels having distal openings in the extradermal surface of the intraosseous appliance that is not the proximal extradermal surface.

In some embodiments, the method can additionally comprise: receiving, from the at least one group of optical components and/or from the at least one electrode array, information about brain vasculature, electrical activity and electrical impedance. In some such embodiments, the method can additionally comprise: performing a brain-mapping using the received information.

In some embodiments, the positioning of the at least one group of optical components can include positioning the first, second and third groups of optical components. In some embodiments, the positioning of at the least one group of optical components can additionally comprise positioning one or more intracerebral optical components within respective channels having distal openings in a distal base of the intraosseous appliance. In some such embodiments, the one or more intracerebral optical components can be joined to respective intracerebral electrodes.

In some embodiments, the positioning of the at least one group of optical components and/or the further positioning of the at least one electrode array can be performed after the inserting. In some embodiments, at least a part of the positioning of the at least one group of optical components can be initiated before the inserting. In some embodiments, at least part of the further positioning of the at least one electrode array can be initiated before the inserting. In some embodiments, the received information can include hemodynamic responses surveilled by functional near infrared spectroscopy (fNIRS). In some embodiments, the received information can include neuronal responses surveilled by fNIRS.

In some embodiments, the received information can include changes in a concentration of oxyhemoglobin and/or hemoglobin in the vicinity of an optical component. In some embodiments, the received information can include electrical impedance change detected using electrical impedance tomography (EIT).

In some embodiments, the intraosseous appliance can comprise a distal portion formed from a material that is substantially transparent to a least a portion of a 400 nm-1 μ section of an electromagnetic spectrum. In some embodiments, the intraosseous appliance can comprise a distal portion formed to allow passage therethrough of a majority of available electromagnetic radiation in at least a portion of a 400 nm-1 μ section of an electromagnetic spectrum.

In some embodiments, the distal portion can be an integrally formed portion of the intraosseous appliance. In some embodiments, the distal portion can be joined to the distal base of the intraosseous appliance and/or can comprise one or more volumes contiguous to at least some of the respective distal openings of the channels of the first array subset.

In some embodiments, the intraosseous appliance can comprise an extradermal annular flange portion having a maximum outer diameter of the intraosseous appliance. In some such embodiments, the annular flange can comprise two opposing surfaces, a first opposing surface including at least a portion of the extradermal surface that is not the proximal extradermal surface, and/or a second opposing surface including at least a portion of the proximal extradermal surface.

In some embodiments, the method can additionally comprise: positioning a thermal sensor at least partly within a channel of the plurality of channels. In some embodiments, the method can additionally comprise: receiving information about a thermal state of an emitter from a thermal sensor.

In some embodiments, the intraosseous appliance can be a first intraosseous appliance, and the method can additionally comprise: inserting, in a second cranial burr hole, a second intraosseous appliance comprising a plurality of channels having respective proximal openings in a proximal extradermal surface; and/or positioning at least one group of optical components at least partly within respective channels having distal openings in a distal base of the intraosseous appliance, wherein an intracerebral optical component of the second apparatus is configured to detect an emission by an intracerebral optical component of the first apparatus.

In some embodiments, the brain-mapping can be based upon information from at least two of stereo-electroencephalography (sEEG), EIT performed within a lower range of two non-overlapping frequency ranges, EIT performed within an upper range of the two non-overlapping frequency ranges, and fNIRS. In some embodiments, the brain-mapping can be based upon information from at least three of stereo-electroencephalography (sEEG), EIT performed within a lower range of two non-overlapping frequency ranges, EIT performed within an upper range of the two non-overlapping frequency ranges, and fNIRS. In some embodiments, the brain-mapping can be based upon information from stereo-electroencephalography (sEEG), EIT performed within a lower range of two non-overlapping frequency ranges, EIT performed within an upper range of the two non-overlapping frequency ranges, and fNIRS.

In some embodiments, the first (lower) range of the two non-overlapping frequency ranges can be below an upper threshold of 500 Hz, or 1 kHz, or 1.5 kHz, or 2 kHz. In some embodiments, the second (upper) range of the two non-overlapping frequency ranges can be above a lower threshold of 3 kHz, or 2 kHz, or 1 kHz.

In some embodiments, the information from fNIRS can include fNIRS information relating to multiple frequencies.

In some embodiments, the method can be carried out so as to perform a monitoring of the brain. In some embodiments, the method can be carried out so as to obtain a localization of one or more epileptogenic foci. In some embodiments, the method can be carried out so as to obtain a characterization of neuro-vascular coupling.

According to embodiments of the presently disclosed subject matter, a system for performing brain mapping comprises: (a) a plurality of apparatuses, each comprising an intraosseous appliance comprising a hollow-channel array that includes a plurality of channels having respective proximal openings in a proximal extradermal surface of the intraosseous appliance, the hollow-channel array comprising at least one array subset selected from: (i) a first array subset comprising a plurality of channels adapted for respective passage therethrough of (A) a first group of optical components including at least one emitter element and at least one detector element, and (B) a first electrode array comprising at least one electrode, the channels of the first array subset having respective distal openings in a distal base of the intraosseous appliance that is opposite the proximal surface, (ii) a second array subset comprising a plurality of channels adapted for respective passage therethrough of a second group of optical components including at least one emitter element and at least one detector element, the channels of the second array subset having respective distal openings in a lateral surface of the intraosseous appliance, and (iii) a third array subset comprising a plurality of channels adapted for respective passage therethrough of (A) a third group of optical components including at least one emitter element and at least one detector element, and (B) a second electrode array comprising at least one electrode, the channels of the third array subset having respective distal openings in an extradermal surface of the intraosseous appliance that is not the proximal extradermal surface. The system additionally comprises: (b) electronic circuitry configured to be in at least one-way communication with, and receive signals from, one or more of the first, second and third groups of optical components, and from one or more of the first and second electrode arrays.

According to embodiments of the presently disclosed subject matter, a system for performing brain mapping comprises: (a) a plurality of apparatuses, each apparatus comprising: (i) an intraosseous appliance shaped for insertion in, and removal from, a cranial burr hole, the intraosseous appliance comprising a plurality of channels having respective proximal openings in a proximal extradermal surface of the intraosseous appliance, (ii) at least one group of optical components, each of the optical components comprising one of an emitter and a detector, the at least one group selected from: (A) a first group of optical components adapted to reside at least partly within respective channels having distal openings in a distal base of the intraosseous appliance so as to establish respective optical paths between respective distal ends of the first-group optical components and a dura of the subject, (B) a second group of optical components adapted to reside at least partly within respective channels having distal openings in a lateral surface of the intraosseous appliance so as to establish respective optical paths between respective distal ends of the second-group optical components and a bone wall of the burr hole, and (C) a third group of optical components adapted to reside at least partly within respective channels having distal openings in an extradermal surface of the intraosseous appliance that is not the proximal extradermal surface so as to establish respective optical paths between respective distal ends of the third-group optical components and skin of the subject, and (iii) an electrode array including at least one electrode subarray selected from: (A) a first electrode subarray comprising one or more intracerebral electrodes adapted to reside at least partly within respective channels having distal openings in a distal base of the intraosseous appliance, and (B) a second electrode subarray comprising one or more extracerebral electrodes adapted to reside at least partly within respective channels having distal openings in the extradermal surface of the intraosseous appliance that is not the proximal extradermal surface. The system also comprises: (b) electronic circuitry configured to be in at least one-way communication with, and receive signals from, one or more of the first, second and third groups of optical components, and from one or more of the first and second electrode arrays.

In some embodiments, each apparatus can comprise the first, second and third groups of optical components. In some embodiments, each apparatus can additionally comprise one or more intracerebral optical components. In some embodiments, each apparatus can additionally comprise one or more intracerebral optical components joined to respective intracerebral electrodes.

In some embodiments, at least some of the respective optical components of each apparatus can be configured for functional near infrared spectroscopy (fNIRS) surveillance of hemodynamic responses. In some embodiments, at least some of the respective optical components of each apparatus can be configured for fNIRS surveillance of neuronal responses. In some embodiments, at least some of the respective optical components of each apparatus can be configured for measuring a change a concentration of oxyhemoglobin and/or hemoglobin in the vicinity of the at least some of the optical components.

In some embodiments, at least some of the electrodes of the respective electrode array of each apparatus can be configured for detecting and imaging electrical impedance change using electrical impedance tomography (EIT). In some such embodiments, the at least some of the electrodes can be configured to perform EIT in at least two non-overlapping frequency ranges. In some embodiments, a first (lower) frequency range can be below an upper threshold of 500 Hz, or 1 kHz, or 1.5 kHz, or 2 kHz. In some embodiments, the second (upper) range of the two non-overlapping frequency ranges can be above a lower threshold of 3 kHz, or 2 kHz, or 1 kHz.

In some embodiments, the at least some of the electrodes can be configured for monitoring an electric field.

In some embodiments, each apparatus can comprise a thermal sensor arranged to monitor a temperature of an emitter.

In some embodiments, each of the apparatuses can be in an assembled state, e.g., such that the optical components of each respective at least one group of optical components are at least partly resident in the respective channels. In some embodiments, each of the apparatuses can be in an assembled state, e.g., such the respective at least one of the electrodes is at least partly resident in the respective channels.

In some embodiments, it can be that (i) each apparatus comprises an intracerebral optical component, and/or (ii) an intracerebral optical component of a first apparatus is configured to detect an emission by an intracerebral optical component of a second apparatus.

In some embodiments, the electronic circuitry can comprise (i) one or more processors; and/or (ii) a computer-readable medium storing program instructions that, when executed by the one or more processors, cause the one or more processors to receive, from the respective at least one group of optical components of at least one apparatus, and from the respective electrode array of at least one apparatus, information about brain vasculature, electrical activity and/or impedance. In some embodiments, the computer-readable medium can additionally store program instructions that, when executed by the one or more processors, cause the one or more processors to perform a brain-mapping using the received information.

In some embodiments, the received information can include hemodynamic responses surveilled by functional near infrared spectroscopy (fNIRS). In some embodiments, the received information includes neuronal responses surveilled by fNIRS. In some embodiments, the received information can include changes in a concentration of oxyhemoglobin and/or hemoglobin in the vicinity of an optical component. In some embodiments, the received information can include electrical impedance change detected using electrical impedance tomography (EIT).

In some embodiments, the computer-readable medium can additionally store program instructions that, when executed by the one or more processors, cause the one or more processors to receive information about a thermal state of an emitter from a thermal sensor. In some embodiments, the computer-readable medium can additionally store program instructions that, when executed by the one or more processors, cause the one or more processors to receive information from the intracerebral optical component of the first apparatus about a detected emission of the intracerebral optical component of the second apparatus.

In some embodiments, the brain-mapping can be based upon information from at least two of stereo-electroencephalography (sEEG), EIT performed within a lower range of two non-overlapping frequency ranges, EIT performed within an upper range of the two non-overlapping frequency ranges, and fNIRS. In some embodiments, the brain-mapping can be based upon information from at least three of stereo-electroencephalography (SEEG), EIT performed within a lower range of two non-overlapping frequency ranges, EIT performed within an upper range of the two non-overlapping frequency ranges, and fNIRS.

In some embodiments, the brain-mapping can be based upon information from stereo-electroencephalography (sEEG), EIT performed within a lower range of two non-overlapping frequency ranges, EIT performed within an upper range of the two non-overlapping frequency ranges, and fNIRS. In some embodiments, a first (lower) range of the two non-overlapping frequency ranges can be below an upper threshold of 500 Hz, or 1 kHz, or 1.5 kHz, or 2 kHz. In some embodiments, wherein a second (upper) range of the two non-overlapping frequency ranges can be above a lower threshold of 3 kHz, or 2 kHz, or 1 kHz. In some embodiments, the information from fNIRS can include fNIRS information relating to multiple frequencies.

The scope of the embodiments includes the electronic circuitry disclosed in accordance with any one or more of the disclosed embodiments.

### BRIEF DESCRIPTION OF THE DRAWINGS

The presently disclosed subject matter will now be described further, by way of example, with reference to the accompanying drawings, in which the dimensions of components and features shown in the figures are chosen for convenience and clarity of presentation and not necessarily to scale. In the drawings:

FIG. 1A is a schematic illustration of an exemplary apparatus for use in brain-monitoring and/or brain-mapping, according to embodiments of the presently disclosed subject matter.

FIG. 1B is a schematic in situ illustration of an exemplary apparatus similar to the apparatus of FIG. 1A, showing respective hollow channels and a representative sample of optical components and electrodes passing therethrough, according to embodiments of the presently disclosed subject matter.

FIG. 1C is a schematic illustration of an alternative design to the exemplary apparatus of FIG. 1A, according to embodiments of the presently disclosed subject matter.

FIG. 2 is a schematic top-view illustration of an intraosseous appliance comprising a plurality of hollow channels, according to embodiments of the presently disclosed subject matter.

FIGS. 3A and 3B are respective side-view and top-view schematic illustrations of an exemplary apparatus comprising an intraosseous appliance, according to embodiments of the presently disclosed subject matter.

FIG. 4 is a schematic illustration of an exemplary apparatus comprising an intraosseous appliance and an intracerebral electrode rotated relative to a longitudinal axis of the appliance, according to embodiments of the presently disclosed subject matter.

FIG. 5 is a schematic in situ cross-sectional view of an exemplary apparatus comprising an intraosseous appliance, according to embodiments of the presently disclosed subject matter.

FIG. 6A is a schematic in situ illustration of a system comprising a plurality of apparatuses comprising respective intraosseous appliances, and electronic circuitry, according to embodiments of the presently disclosed subject matter.

FIG. 6B includes a more detailed representation of the electronic circuitry of FIG. 6A, according to embodiments of the presently disclosed subject matter.

FIGS. 7, 8A, 8B, 8C, 8D, 8E, and 8F show flowcharts of methods and method steps for monitoring a human brain, according to embodiments of the presently disclosed subject matter.

FIG. 9 shows a system for brain monitoring and/or brain mapping in accordance with embodiments of the present disclosure.

FIG. 10 illustrates an enlarged view of a part of FIG. 9.

FIG. 11 illustrates two isometric views of an intraosseous appliance according to embodiments of the present disclosure.

### DETAILED DESCRIPTION OF THE ILLUSTRATED EMBODIMENTS

The presently disclosed subject matter is herein described, by way of example only, with reference to the accompanying drawings. With specific reference now to the drawings in detail, it is stressed that the particulars shown are by way of example and for purposes of illustrative discussion of the preferred embodiments of the presently disclosed subject matter only, and are presented in the cause of providing what is believed to be the most useful and readily understood description of the principles and conceptual aspects of the presently disclosed subject matter. In this regard, no attempt is made to show structural details of the presently disclosed subject matter in more detail than is necessary for a fundamental understanding of the presently disclosed subject matter, the description taken with the drawings making apparent to those skilled in the art how the several forms of the presently disclosed subject matter may be embodied in practice. Throughout the drawings, like-referenced characters are generally used to designate like elements. Subscripted reference numbers (e.g., 10₁) or letter-modified reference numbers (e.g., 100_{A}) are used to designate multiple separate appearances of elements in a single drawing, e.g. 10₁ is a single appearance (out of a plurality of appearances) of element 10, and 100_{A} is a single appearance (out of a plurality of appearances) of element 100, and/or disclosed variations in like elements.

Embodiments disclosed herein relate to the use of multiple techniques for monitoring and mapping brain activity, and to systems and apparatuses for carrying out such techniques. Some embodiments relate to a hybrid system that improves the ability to localize epileptogenic foci and characterize neuro-vascular coupling during ictal, preictal and interictal periods. According to embodiments, electrodes and optical components are integrated in one intraosseous appliance inserted into the brain and skull of patients with epilepsy, to directly measure the brain vasculature, electrical activity and impedance using EIT (electrical impedance tomography), enabling better localization of ictal and interictal epileptic activities while substantially reducing motion artifacts and contamination from extracerebral factors. In systems comprising the intraosseous appliance, optical components and electrodes, multi-modal software is provided to co-register the signals received from the “optrodes” (optodes, i.e., optical components, +electrodes) with whole-brain imaging and mapping.

In embodiments relating to monitoring, measuring and mapping brain functions and activity, one or more modalities are employed for the purpose, selected from, but not exhaustively: stereo-electroencephalography (SEEG), high-frequency electrical impedance tomography (hfEIT), low-frequency impedance tomography (lfEIT), functional near infrared spectroscopy fNIRS), intracranial pressure, oxygen tension and blood flow. In a non-limiting example, and without subscribing to a particular theory, SEEG is effective with respect to phase-synchronized neuronal activity, hfEIT is effective with respect to cellular and hemodynamic processes, and in particular blood and extracellular fluid volume, lfEIT is effective with respect to cellular and hemodynamic processes, and in particular blood and extracellular fluid volume and cellular processes, and fNIRS is effective with respect to hemodynamic processes.

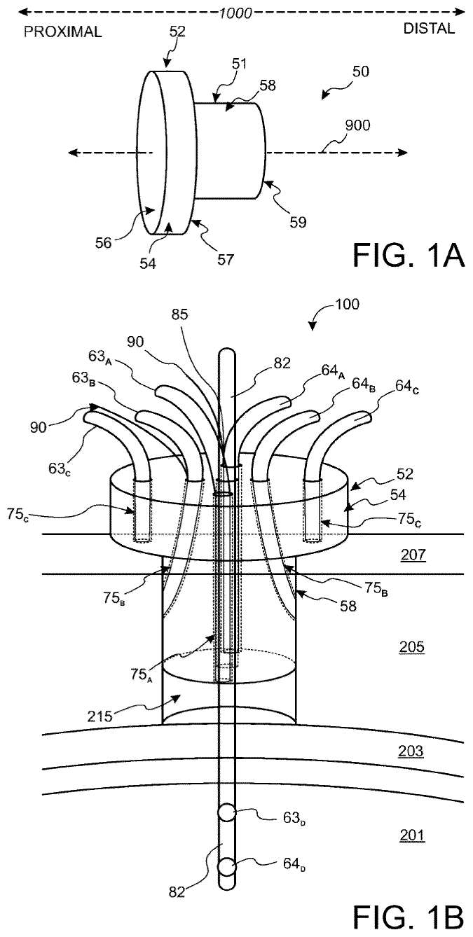

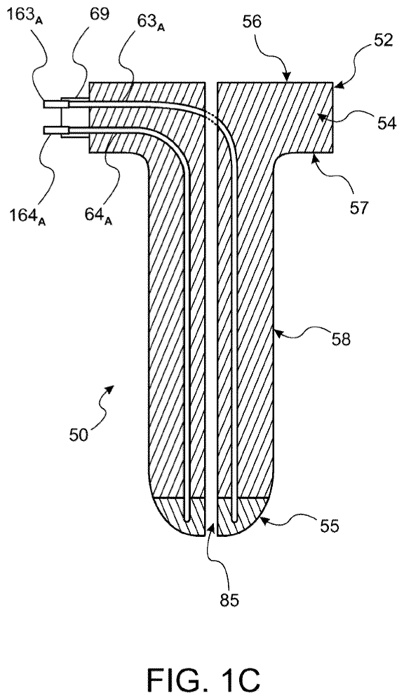

We now refer to the figures, and in particular to FIGS. 1A, 1B and FIG. 11. In the following, the reference number 75 may be used to refer to any of the elements referred to as 75_{A}, 75_{B} or 75_{C}.

FIG. 1A is a schematic illustration of an appliance 50 configured for use in brain-monitoring and brain-mapping, in particular for measuring brain vasculature, electrical activity and impedance using EIT. The appliance 50 is shown in FIG. 1A on its side, i.e., a central longitudinal/vertical axis is indicated by arrow 900. Respective proximal and distal directions as used herein are shown by arrow 1000. In one non-limiting intended use of the appliance 50, the appliance 50 and the longitudinal/vertical axis indicated by arrow 900 are oriented vertically, and the terms ‘upper’ and ‘lower’ are used with reference to the axis 900. In other non-limiting intended uses, the axis 900 is rotated in accordance with a shape of a subject's head, such that the ‘vertical’ axis is (or is close to) a normal vector to the user's head and, and the terms ‘upper’ and ‘lower’ correspond to ‘further from the center of the head’ and ‘closer to the center of the head’ in such intended uses.

The appliance 50 of FIG. 1A comprises a vertical body 51 shown for convenience as having a generally cylindrical shape. In other examples (not illustrated), the vertical body 51 can have a cross-section that is oval, elliptical, square, or any other practical shape. The vertical body 51 comprises a proximal upper surface 56, a lateral surface 58, and a distal ‘base’ surface 59. In the non-limiting example of FIG. 1A, the appliance 50 comprises an upper annular flange portion 52 that has the maximum outer diameter of the intraosseous appliance 50, e.g., a diameter larger than the diameter of the vertical body 51. The upper surface of the flange portion 52 can be coplanar with the upper surface 56 of the vertical body 51, as shown, or can be higher or lower. In some embodiments, the flange portion 52 can be integrally formed with the vertical body 51, while in other embodiments the flange portion 52 can comprise a separate element joined, e.g., by heat and/or pressure and/or adhesives, to the vertical body 51. A non-limiting example of a flange portion 52 includes a distal surface 57, which is annular to the vertical body, and a lateral surface 54 that is distinct from the lateral surface 58 of the vertical body 51. In other examples, the flange portion 52 may not have a distinct lower (distal) surface 57, and the outer perimeter of the flange can slope down to the vertical body 51. In such examples, the lateral surface 54 of the flange portion 52 merges with the lateral surface 58 of the vertical body 51. Respective aspect ratios and relative sizes of the vertical body 51 and of the flange portion 52 can be chosen by the skilled designer in accordance with the functionality and physical interfaces of the appliance 50, which are described in further detail hereinbelow.

FIG. 1B shows the appliance 50 of FIG. 1A incorporated in an apparatus 100 and disposed in situ, i.e., with the appliance 50 deployed as follows: the vertical body 51 sits at least partly disposed in a burr hole 215 formed in a human subject's skull 205; an upper portion of the vertical body 51 is disposed within the subject's scalp 207; and the distal ‘base’ surface 59 is disposed within the skull 205. The flange portion 52 is entirely external to the scalp, such that both the proximal upper surface 56 (of the vertical body 51 and of the flange portion 52) and the distal surface 57 of the flange portion 52 are extradermal.

As can be seem in FIG. 1B, the appliance 50 includes a number of hollow channels 75, 85 having proximal openings on the proximal upper surface 56, and distal openings as follows: a first group of channels 75 has distal openings in the distal base surface 59 of the appliance 50 (i.e., of the vertical body 51), and includes channels 75_{A} for passage therethrough of respective optical components including emitters 63_{A} and detectors 64_{A}, and one or more channels 85 for passage therethrough of respective electrodes, e.g., depth electrodes 82; a second group of channels 75_{B} (also referred to herein as skull wall optode channels) for passage therethrough of respective optical components including emitters 63_{A} and detectors 64_{A}, has distal openings in the lateral surface 58 of the vertical body 51 in a section of the vertical body 51 that is designed to be deployed intraosseously, i.e., within the thickness of the skull 205; and a third group of channels has distal openings in the distal surface 57 of the flange portion 52 and includes channels 75_{C} for passage therethrough of respective optical components including emitters 63_{C} and detectors 64_{C}, and one or more channels 85 for passage therethrough of respective electrodes. Either of the terms “emitter” and “detector” as used herein can mean either an assembly including the actual functional components attached to a wire or fiber, or the actual functional components themselves.

FIG. 11 illustrates the appliance 50 in some other embodiments. As can be seen, the appliance 50 includes a number of hollow channels having proximal openings on the proximal upper surface, and distal openings as follows: a first group of channels has distal openings in the distal base surface of the appliance 50, and includes channels 75_{A} (also referred to herein as brain optode channels) for passage therethrough of respective optical components including emitters and/or detectors, and one or more channels 85 for passage therethrough of respective electrodes, e.g., depth electrodes. The appliance 50 includes also a group of channels having distal openings in a distal surface of a flange portion and includes channels 75_{C} (also referred to herein as extradermal optode channels) for passage therethrough of respective optical components including emitters and detectors,

The skilled artisan will understand that in embodiments (not illustrated) in which the appliance 50 does not include or is not enhanced by a flange portion 52, the appliance is designed to be disposed in situ such that an upper portion of the vertical body 51 remains external to the scalp 207 while a lower portion is installed intraosseously, and the third group of channels 75_{C} has distal openings in the corresponding upper portion of the lateral surface 58 that remains external to the scalp 207, i.e., not in a distinct lower flange surface 57.

An apparatus 100 according to embodiments, as illustrated schematically in an assembled state in FIG. 1B, includes the appliance 50, corresponding optodes 63, 64 (emitter elements 63 and detector elements 64), corresponding electrode(s) 82, and one or more sensors such as a thermal (e.g., temperature) sensor 90, an intercranial pressure sensor (not shown), an oxygen tension sensor (not shown), and a blood-flow sensor (not shown). The assembled state is such that the optodes, electrode(s) and sensor(s) are at least partly resident in corresponding hollow channels.

In embodiments, fNIRS (functional near infrared spectroscopy) is used to monitor and/or record the hemodynamic response of the brain tissue to epileptic seizures. The portion of the electromagnetic spectrum that is of interest is the 400 nm-1 μ range. As is known, fNIRS is an imaging technique based on the different near infra-red light absorption properties of oxyhemoglobin (HbO₂) and deoxyhemoglobin (Hb). Since brain tissue activity is associated with increased arterial blood supply, the dynamics of relative HbO₂ and Hb concentrations act as a surrogate of brain tissue activity that can be detected by optical components using fNIRS. The optical components (optodes), comprise respective light emitters and detectors placed in contact directly or indirectly with the tissue of interest. The optodes can comprise optical fibers, or bundles or arrays of optical fibers, which are connected to light sources 63, such as LEDs or lasers, and to light detectors 64, such as photodiodes, cameras, photon counters etc. Alternatively, the light sources and detectors 64 can form the optodes directly without fibers. In order to measure changes in the concentrations of HbO₂ and Hb, the emitter elements 63 are configured to emit at least two wavelengths of light.

Still referring to FIG. 1B, a first group of optodes—emitters 63_{A} and detectors 64_{A}—are configured for passage through channels 75_{A}, which, in the assembled state of FIG. 1B, are disposed with proximal ends of the optodes reaching and/or extending through respective proximal openings of the channels 75_{A} on the upper surface 56, and distal ends of the optodes reaching and/or extending through respective distal openings of the channels 75_{A} on the distal base 59. The arrangement of the first group of optodes thus positions the functional components of the emitters 63_{A} and detectors 64_{A} so as to establish respective optical paths between the functional components and the brain 201 of the subject. The phrase ‘optical path’ means direct and/or indirect optical communication, i.e., the intervening layer(s) between emitter and detector, e.g., the dura 203, do not completely block measuring and monitoring therethrough. Thus, the functional components of the emitters 63_{A} and/or detectors 64_{A} can be disposed within the skull 205 but still be capable of indirect optical communication therethrough.

A second group of optodes—emitters 63_{B} and detectors 64_{B}, are configured for passage through channels 75_{B}, which, in the assembled state of FIG. 1B, are disposed with proximal ends of the optodes reaching and/or extending through respective proximal openings of the channels 75_{B} on the upper surface 56, and distal ends of the optodes reaching and/or extending through respective distal openings of the channels 75_{B} on the lateral surface 58 of the vertical body 51. The arrangement of the second group of optodes thus positions the functional components of the emitters 63_{B} and detectors 64_{B} so as to establish respective optical paths between the functional components and the bone wall of the burr hole 215 of the subject.

A third group of optodes—emitters 63_{C} and detectors 64_{C}, are configured for passage through channels 75_{C}, which, in the assembled state of FIG. 1B, are disposed with proximal ends of the optodes reaching and/or extending through respective proximal openings of the channels 75_{A} on the upper surface 56, and distal ends of the optodes reaching and/or extending through respective distal openings of the channels 75_{C} on the distal surface 57 of the flange portion 52 (if present, as discussed above with respect to channels 75_{C}). The arrangement of the third group of optodes thus positions the functional components of the emitters 63_{C} and detectors 64_{C} so as to establish passage of light through the outer surface (skin) of the scalp 207.

FIG. 1B has been simplified for making details discernible; in some examples, the quantities of hollow channels 75, 85, optodes 63, 64 and depth electrodes 82, are different than shown in FIG. 1B, e.g., greater than shown in FIG. 1B.

In embodiments, stereo-electroencephalography (SEEG) is used to record electroencephalographic signals via one or more depth electrodes 82, at least partly resident in hollow channel 85, which comprise bundles of isolated cables or wires (e.g., between 8 and 16), each of them ending in a metallic contact, e.g., a platinum-iridium contact. The size of the contact can vary. In non-limiting examples, the diameter is between 0.5 and 1.5 mm (all ranges cited herein are inclusive), or between 0.7 and 0.9 mm, or about 0.8 mm. The distance between contacts is generally less than 10 mm, or less than 0.8 mm, or about 5 mm. These electrodes can be implanted bilaterally and can be placed in different structures of the brain 201: insula, hippocampus, mesial frontal area, and others.

FIG. 1C shows certain features of an apparatus according to an alternative structural design of the appliance 50. In the alternative design of FIG. 1C, some optical emitters 63 and detectors 64 reside partly in channels 75 (not shown in FIG. 1C) which have proximal openings on a lateral surface 54 of the flange portion 52 and not on an upper surface 56. The lateral surface 54 of the flange portion 52, when the appliance 50 is disposed in situ, is extradermal and faces away from the appliance much like the upper surface 56 such that the functionality of having the proximal channel opening in the lateral surface 54 provides, in most implementations, the same functionality as having the proximal channel opening in the upper surface 56. In the non-limiting example of FIG. 1C, an emitter 63_{A} and a detector 64_{A} pass through openings of channels in the lateral surface 54 of the flange portion 52, where they connect to optical fibers 163_{A}, 164_{A}, respectively at optical connector 69. In embodiments, the emitter 63_{A} and detector 64_{A} belong to the first group of optodes in which distal ends of the optodes reach and/or extend through respective distal openings of the channels on the distal base 59. In additional or alternative examples according to this design (not illustrated), optodes of the second group of optodes 63_{B}, 64_{B}, in which distal ends of the optodes reach and/or extend through respective distal openings of the channels 75_{B} on the lateral surface 58 of the vertical body 51 may similarly reside in channels 75_{B} opening proximally in the lateral surface 54 of the flange portion 52 rather than in the upper surface 56. In further additional or alternative examples according to this design (not illustrated), optodes of the third group of optodes 63_{C}, 64_{C}, in which distal ends of the optodes reach and/or extend through respective distal openings of the channels 75_{C} on the distal surface 57 of the flange portion 52 (if present) may similarly reside in channels 75_{C} opening proximally in the lateral surface 54 of the flange portion 52 rather than in the upper surface 56. In still further additional or alternative examples, any one or more of depth electrodes 82, EIT electrodes 87, 88, and temperature sensors 90 (or other optional sensors such as an intercranial pressure sensor, an oxygen tension sensor, or a blood-flow sensor) may similarly reside in channels opening proximally in the lateral surface 54 of the flange portion 52 and not in the upper surface 56.

With respect to any feature disclosed herein describing optodes and/or electrodes and/or sensors passing through a channel opening in an upper proximal surface of the appliance, the feature applies equally, mutatis mutandis, to the alternative structural example of FIG. 1C in which the optodes and/or electrodes and/or sensors passing through a channel opening in a lateral surface of the flange portion of the appliance, and for the sake of conciseness, no further effort is made to show or describe the alternative structural design of FIG. 1C.

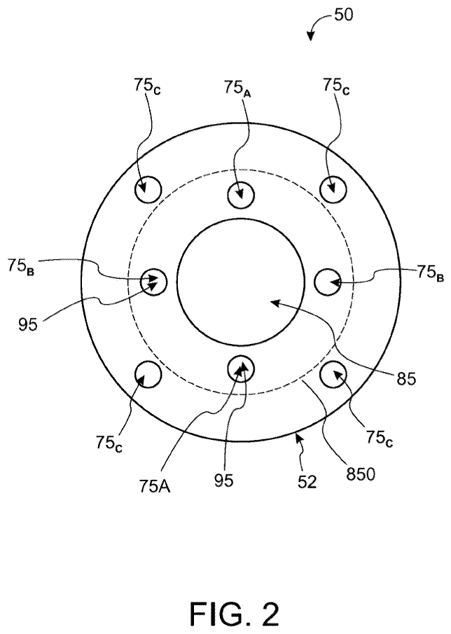

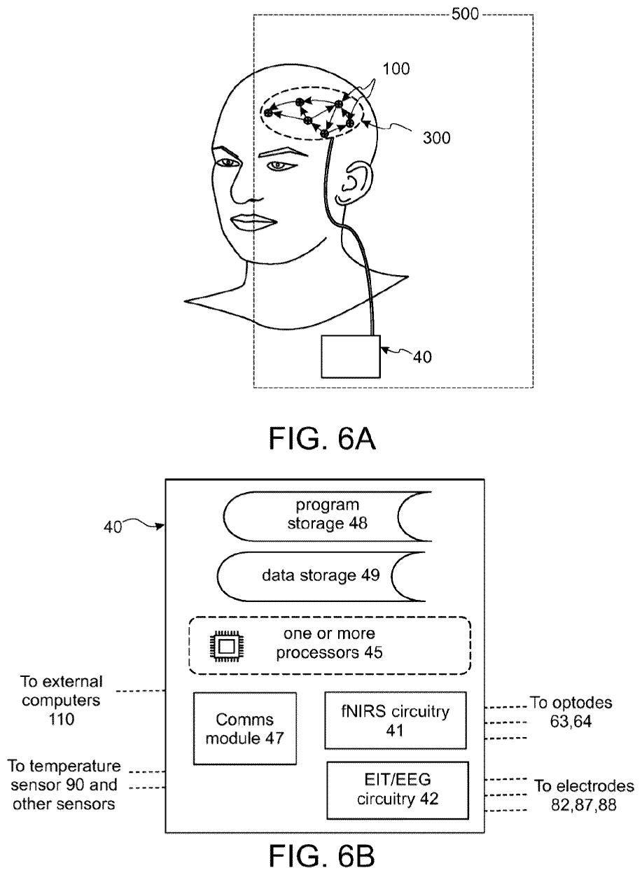

FIG. 2 is a schematic top view of an appliance 50 similar to the appliances 50 of FIGS. 1A and 1B, showing the upper surface 56. Dotted line 850 indicates the footprint of the vertical body 51 within the footprint of the flange portion 52. Channels 75_{C}, which have respective distal openings on the distal surface 57 of the flange portion 52, have respective proximal opening within the annularly differential footprint of the flange portion 52. Channels 75_{A} and 75_{B}, which have respective distal openings in the base 59 and lateral wall 58 of the vertical body 51, have respective proximal openings in the portion of the proximal (upper) surface 56 corresponding to the footprint of the vertical body 51. Hollow channels designated for passage therethrough of thermal sensors 90 and/or other sensors are shown as channels 95. In the non-limiting example of FIG. 2, the same channel can be both an optode-carrying channel 75 and a sensor-carrying channel 95. A thermal sensor can thus be co-resident in a channel with, e.g., an emitter optode so as to monitor the temperature of the emitter and report that information to a control system, e.g., electronic circuitry 40 of FIGS. 6A and 6B.

According to embodiments, EIT is used for detecting and imaging changes in electrical impedance in different parts of the brain. EIT of the brain is sensitive to both hemodynamic and cellular (neuronal and glial processes). Using intracranial electrodes, EIT can record electrical impedance change associated with epileptic activity.

The use of EIT, according to embodiments, is based on passing alternating current (AC) through a pair of triggering electrodes and sensing the electric field associated with this current by a number of other electrodes, e.g., recording electrodes. The frequency of the AC current is preferably set far from the frequency spectrum of biological electrical activity. In an example, the current has a frequency range in the tens of kilohertz, in which case the EIT is effective to measure resistance, but not necessarily capacitance, and therefore is particularly sensitive to changes in volume of blood and extracellular fluid. In another example the AC current has a much lower frequency range, e.g., about 1 kHz, where the EIT is sensitive also to cellular processes. In some embodiments, EIT is used in two different frequency ranges, known as high-frequency EIT (hfEIT) and low-frequency EIT (lfEIT). In a first non-limiting example, a first frequency range is below an upper threshold of 500 Hz, or 1 kHz, or 1.5 kHz, or 2 kHz, and a second frequency range is above a lower threshold of 3 kHz, or 2 kHz, or 1 kHz. In a second non-limiting example, a first frequency range is below an upper threshold of 250 Hz, or 500 Hz, or 1 kHz, or 1.5 kHz, or 2 kHz, and a second frequency range is above a lower threshold of 5 kHz, or 10 kHz, or 15 kHz, or 20 kWhz.

During epileptic activity, neuronal swelling due to electrolyte and water entrance into neurons tends to reduce extracellular fluid volume and increase electrical resistance. Later, with hemodynamic response, increasing blood volume tends to decrease electrical resistance, and thus the cellular and hemodynamic processes can overlap and not easily be distinguished from each other by EIT only. It can therefore be desirable to combine NIR optodes, e.g., optodes operating in an NIR range of 680-1100 nm or at higher or lower wavelengths, together with EIT electrodes, and incorporate them into a single appliance.

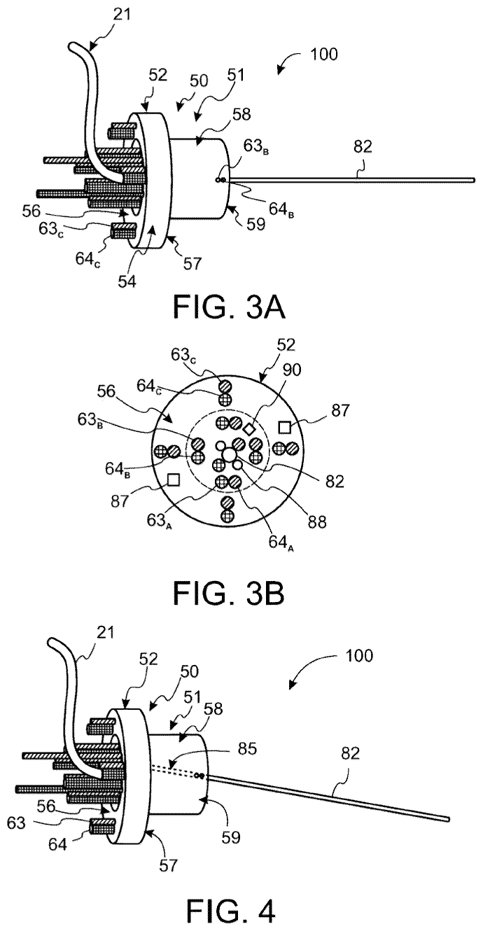

Referring now to FIGS. 3A and 3B, an apparatus 100 according to embodiments includes an appliance 50, e.g., an intraosseous appliance, pairs of optodes 63_{A-C}, 64_{A-C}, one or more depth electrodes 82 (with electrical lead 21), EIT electrodes (contacts 88 and trigger(s) 87), and a thermal sensor 90. Additional optional sensors (not shown) include an intercranial pressure sensor, an oxygen tension sensor, and a blood-flow sensor. The apparatus 100 is shown in an assembled state in which the first, second and third groups of optodes 63, 64 described above are resident in respective first, second and third channels 75 described above. of the first, second and third array subsets. The apparatus 100 can be provided as a kit, whether in an assembled state as shown in FIGS. 3A and 3B, or in an unassembled state.

In embodiments, the distal ends of both the optodes 63, 64 and the EIT electrodes 87, 88 are placed on both sides of the scalp and calvarium. Inter alia, this enables separation of signals originating from extracranial vs. intracranial compartments (e.g., from scalp-based signals vs. brain-based signals).

FIG. 4 illustrates an example of an apparatus 100 in which the channel 85 configured for passage therethrough of the depth electrode 82 traverses the appliance 50 from a respective proximal opening in the proximal extradermal surface 56 to a respective distal opening in the distal base 59 at an angle of at least 5° from a central longitudinal axis of the appliance (arrow 900 of FIG. 1A), or at an angle of at least 10° therefrom, or at an angle of at least 15° therefrom, or at an angle of at least 20° therefrom. Thus, when in situ, the depth electrode 82 is aligned to enter the brain 201 at an angle.

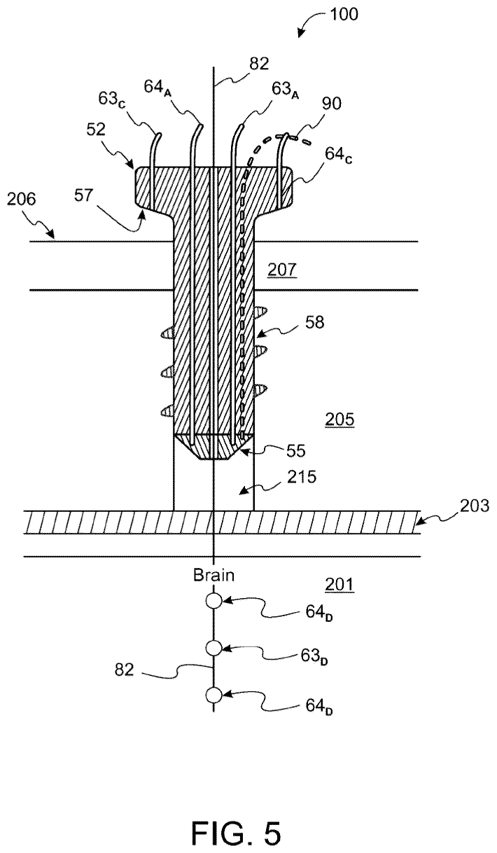

We now refer to FIG. 5, a schematic cross-section of an exemplary apparatus in situ, i.e., with a portion of the appliance disposed intraosseously. In embodiments, the use of intracerebral optodes, especially detectors 64_{D}, but additionally or alternatively emitters 63_{D}, can enhance ability to detect intracerebral hemodynamic changes. Such intracerebral optodes can be integrated into the design of depth electrodes 82. Thus, in FIG. 5, one or more intracerebral optical components (detectors 64_{D} or emitters 63_{D}) are joined to the intracerebral portion of the depth electrode 82. While only a partial sample of optodes are shown in FIG. 5, a pair of optodes (emitter 63_{C} and detector 64_{C}) is shown to pass through respective channels 75_{C}, not shown in FIG. 5, from a proximal surface 56 of the flange portion 52 of the appliance 50 to a distal, yet extradermal surface 57 of the flange portion, and thus are positioned to face the skin 206 of the subject's scalp 207. Another pair of optodes (emitter 63_{A} and detector 64_{A}) is shown to pass through respective channels 75_{A} (not shown in FIG. 5) from the proximal surface of 56 of the flange portion 52 of the appliance 50 to, and through a distal opening in the distal base 59 of the appliance 50.

In the non-limiting example of FIG. 5, the appliance 50 comprises a distal portion 55 below the distal base 59. In some other designs the distal portion 55 is joined to the distal base 59 of the appliance 50, and includes therein one or more volumes contiguous one or more distal-base openings of channels 75_{A}. It can be desirable for the distal portion 55 to be formed of a material that is substantially transparent or diffusing in at least a portion of a 400 nm-2 μ (visible plus near-infrared) section of the electromagnetic spectrum. In embodiments, the distal portion 55 is formed to allow passage therethrough of electromagnetic radiation of at least one wavelength in the 400 nm-2 μ section of the electromagnetic spectrum. A non-limiting example of a suitable material for fabrication of the distal portion 55 is a transparent or partially transparent polymer such as polymethyl methacrylate (PMMA). The vertical body 51 of the appliance 50 can be formed, for example, from a rigid polymer or electrically isolating, non-ferromagnetic metal or metal alloy. Inter alia, this enables efficient EIT measurements on both sides of the scalp 207, and implementation of external magnetic sensors. In a non-limiting example, the intraosseous appliance is formed of a NIR-transparent polymer. In some designs, the distal portion 55 is an integrally formed portion of the appliance 50.

Still referring to FIG. 5, the lateral surface 58 of the vertical body 51 is, in some embodiments, provided with a threading, e.g., to facilitate intraosseous placement of the appliance 50 in the skull 205 (and removal therefrom), and/or to better secure the appliance 50 in the skull for the duration of any brain monitoring.

In embodiments, an array comprising multiple intraosseous appliances with respective optodes and electrodes can be inserted into the skull, thus enabling, e.g., monitoring and recording of signals (electric field, electrical impedance, optic signals) from large, confluent areas of the brain, including detection at one appliance of signals emitted or generated at another intraosseous appliance. FIG. 6A is a schematic illustration of a system 500 comprising a array 300 of apparatuses 100 according to any of the embodiments described hereinabove, each apparatus 100 comprising an intraosseous appliance 50, and a respective optodes 63, 64, electrodes 82, 87, 88 and sensors (e.g., a thermal sensor 90). The system 500 also includes a control system 40 arranged to receive information such as images and measurements from the various apparatuses 100. In embodiments, the fNIRS detectors 64 in one appliance 50 can sense light that is being emitted by another apparatus 100. The influence of different emitters 63 can be separated, e.g., by time or frequency encoding. In some embodiments, using arrays 300 of apparatuses 100 combining depth electrodes 82 and optodes can be used to successfully monitor the electrical neuronal activity and hemodynamic activity in both the deep parts of the brain (e.g., by depth electrodes 82) and the dorsolateral cortex (e.g., by the combination of depth electrodes 82 and optodes 64).

The term “control system” as used herein means a computing device configured for monitoring, controlling, regulating and/or actuating one or more components, systems or sub-systems. A controller should be understood to include any or all of (and not exhaustively): one or more processors, one or more computer-readable media, e.g., transient and/or non-transient storage media, e.g., media containing program instructions for execution by the one or processors, communications arrangements, one or more power sources and/or a connection to a power source, and firmware and/or software.

Referring now to FIG. 6B, a control system 40 according to embodiments is illustrated schematically to show selected components. The exemplary control system 40 of FIG. 6B includes one or more computer processors 45, computer-readable storage media comprising program storage 48 and data storage 49, a communications module 47, fNIRS circuitry 41 for processing data from optodes 63, 64, and EIT/EEG circuitry 42 for processing data from electrodes 82, 87, 88. The computer-readable storage media 48, 49 can include transient and/or transient storage, and can include one or more storage units, all in accordance with desired functionality and design choices. The program storage 48 can be used for any one or more of: storing program instructions, in firmware and/or software, for execution by the one or more processors 45 of the control system 40. In embodiments, the stored program instructions include program instructions for monitoring a human brain 201, including, inter alia, program instructions for obtaining a localization of one or more epileptogenic foci, and/or to obtain a characterization of neuro-vascular coupling. Data storage 49 is optionally separate from program storage 48 can be provided for historical data, e.g., actual measured and calculated values, imaging data, and other data related to the operation of the system 500. In some embodiments, the two storage modules 48, 49 form a single module. The communications module 47 is configured to establish communications links, e.g., with optical sensors 63, 64, electrodes 82, 87, 88, and sensors such as temperature sensors 90. The communications module 47 is optionally configured for transfer of data to and/or from an external, e.g., local, remote and/or cloud, computer 110. In some embodiments, a control system 150 does not necessarily include all of the components shown in FIG. 6B. The terms “communications arrangements” or similar terms such as “communications links” as used herein mean any wired connection or wireless connection via which data communications can take place. Non-limiting and non-exhaustive examples of suitable technologies for providing communications arrangements include any short-range point-to-point communication system such as IrDA, RFID (Radio Frequency Identification), TransferJet, Wireless USB, DSRC (Dedicated Short Range Communications), or Near Field Communication; wireless networks (including sensor networks) such as: ZigBee, EnOcean; Wi-fi, Bluetooth, TransferJet, or Ultra-wideband; and wired communications bus technologies such as. CAN bus (Controller Area Network, Fieldbus, FireWire, HyperTransport and InfiniBand.

According to embodiments, methods can include placement of an intraosseous appliance 50 in the skull 201, followed by installation of one or more depth electrodes 82. The depth electrode 82 passes through a hollow channel 85 in the appliance 50, which is seated in a burr hole 215 and is fixed to its walls, e.g., by a threaded lateral surface 58. The distal base 59 (lower end) of the appliance 50 can be located up to several millimeters from the dura 203. In embodiments, fNIRS optodes (both emitters 63 and detectors 64) are incorporated into the appliance 50 on different levels: on the skin 206, in contact to the bone walls in the lower part of the burr hole 215, and on the lower face 59 of the appliance 50, emitting light directly above the dura 203.

The upper part 52 of the appliance, which stands above the skin 206 or scalp 207, can have a larger size (i.e., diameter) than the vertical body 51 of the appliance that passes through the scalp 207 and bone 205. Inter alia, this enables stabilization a pair of optodes (emitter 63 and detector 64) on the skin 206 or scalp 207, minimizing motion artifacts. In this situation the appliance can play a role of anchor for tree pairs 63, 64 of fNIRS optodes: upper optodes 63_{C}, 64_{C} on and facing the skin 206, intermediate optodes 63_{B}, 64_{B} facing the bone walls of the burr hole 215, and lower optodes 63_{A}, 64_{A} on the dura 203, i.e., having distal ends facing the dura 203.

Optodes are located both above and beneath the scalp 207. The different optodes can be applied to measure the changes in the concentration of HbO₂ and Hb in the vicinity of the optodes. The skull 205 separates between the intermediate optodes 63_{B}, 64_{B} and lower optodes 63_{A}, 64_{A}, and the scalp 207. In embodiments, this allows separation between the optic signals of the scalp 207 and the brain 205, improving signal-to-noise ratio for the optic brain signals. To control the heating, a thermal sensor 90 (for example, based on optic fiber) can also be incorporated into the appliance 50, close to one or more of the fNIRS emitters 63.

In embodiments, fNIRS optodes 63, 64 are accompanied by triggering or sensing EIT electrodes 87, 88: some of them are in contact with the skin 206, others with the wall of the burr hole 215, and others face the brain 201, e.g., face the dura 203 or pass through the dura 203. Other triggering or sensing EIT electrodes will be the EEG scalp electrodes and depth electrodes 82. Placement of EIT triggering electrodes on both sides of the calvarium create additional degrees of freedom of signal, that serve to help distinguish between extracranial and intracranial processes and thus to assess the depth of brain activity.

According to the methods, simultaneous use of all four modalities—SEEG, lfEIT, hfEIT and fNIRS—are used to help distinguish between cellular and hemodynamic processes, and between phase locked neuronal activities and non-phase locked cellular processes.

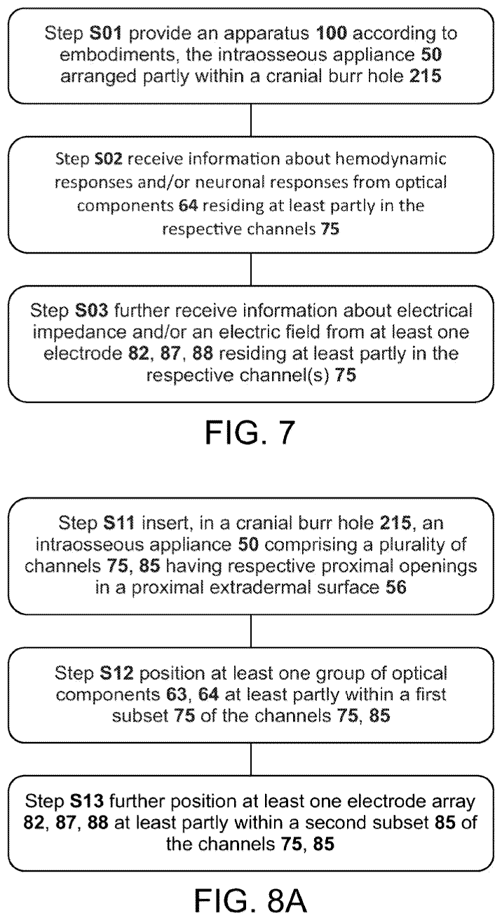

Referring now to FIG. 7, a first method is disclosed for monitoring a human brain 205, using apparatuses 100 according to any one or more of the apparatuses 100 disclosed herein. As illustrated by the flowchart in FIG. 7, the method comprises at least the three method steps S01, S02, and S03:
Step S01: providing the apparatus 100 according to any embodiments disclosed herein, the providing being such that the intraosseous appliance 50 arranged partly within a cranial burr hole 215.
Step S02: receiving information about hemodynamic responses and/or neuronal responses from optical components 64 residing at least partly in the respective channels 75.
Step S03: further receiving information about electrical impedance and/or about an electric field from at least one electrode 82, 87, 88 residing at least partly in the respective channels 75.

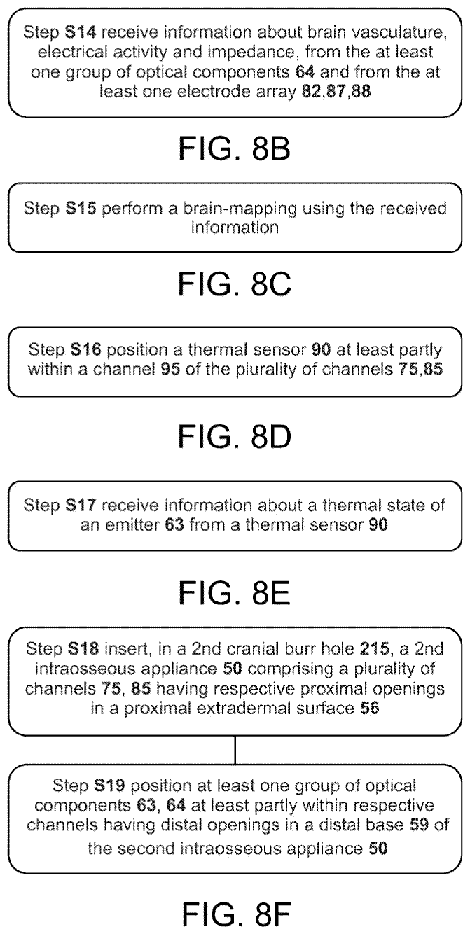

Referring now to FIG. 8A, a second method is disclosed for monitoring a human brain 205. As illustrated by the flowchart in FIG. 8A, the method comprises at least the three method steps S11, S12, and S13:
Step S11: inserting, in a cranial burr hole 215, an intraosseous appliance 50 comprising a plurality of channels 75, 85 having respective proximal openings in a proximal extradermal surface 56.

In some embodiments, the intraosseous appliance 50 comprises a distal portion 55 formed from a material that is substantially transparent to a least a portion of a 400 nm-2 μ section of an electromagnetic spectrum. The intraosseous appliance comprises a distal portion formed to allow passage therethrough of a majority of available electromagnetic radiation in at least a portion of a 400 nm-2 μ section of an electromagnetic spectrum. Such passage may be in the form of scattering or with minimal absorption e.g., an optical diffuser. The distal portion 55 can be an integrally formed portion of the intraosseous appliance 50, or it can be joined to the distal base 59 of the intraosseous appliance 50, in which case it can comprise one or more volumes contiguous to at least some of the respective distal openings on the distal base of the channels 75_{C}, 85.

In some embodiments, the intraosseous appliance 50 comprises an extradermal annular flange portion 52 having the largest maximum outer diameter of the intraosseous appliance 50. The annular flange 52 can comprise two opposing surfaces, a first opposing surface including at least a portion of the extradermal surface 57 that is not the proximal extradermal surface 56, and a second opposing surface including at least a portion of the proximal extradermal surface 56.
Step S12: positioning at least one group of optical components 63, 64 at least partly within a first subset 75 of the channels 75, 85. Each of the optical components comprises at least one of an emitter 63 and a detector 64. The positioning includes at least one of: (i) positioning a at least one first group of optical components 63_{A}, 64_{A} within respective channels 75_{A} having distal openings in a distal base 59 of the intraosseous appliance 50 so as to establish respective optical paths between respective distal ends of the first-group optical components 63_{A}, 64_{A} the brain 201 of the subject, (ii) positioning a second group of optical components 63_{B}, 64_{B} adapted to reside at least partly within respective channels 75_{B} having distal openings in a lateral surface 58 of the intraosseous appliance 50 so as to establish respective optical paths between respective distal ends of the second-group optical components 63_{B}, 64_{B} and a bone wall of the burr hole 215, and (iii) positioning a third group of optical components 63_{C}, 64_{C} adapted to reside at least partly within respective channels 75_{C} having distal openings in an extradermal surface 57 of the intraosseous appliance that is not the proximal extradermal surface 56 so as to establish respective optical paths between respective distal ends of the third-group optical components 63_{C}, 64_{C} and the skin 206 of the subject.

In some embodiments, Step S12 includes positioning the first, second, and third groups of optical components. In some embodiments, Step S12 additionally comprises positioning one or more intracerebral optical components 63_{D}, 64_{D} within respective channels having distal openings in a distal base of the intraosseous appliance. The one or more intracerebral optical components 63_{D}, 64_{D} can be joined to respective intracerebral electrodes 82.
Step S13: further positioning at least one electrode array 82, 87, 88 at least partly within a second subset 85 of the channels 75, 85. The further positioning includes at least one of: (i) positioning a first electrode subarray comprising one or more intracerebral electrodes 82 within respective channels 85 having distal openings in a distal base 59 of the intraosseous appliance 50, and (ii) positioning a second electrode subarray comprising one or more extracerebral electrodes 87, 88 within respective channels having distal openings in the extradermal surface 57 of the intraosseous appliance that is not the proximal extradermal surface 56.

In some embodiments, Steps S12 and S13 are performed after Step S11. In some embodiments, at least a part of Step S12 and/or a part of Step S13 is initiated before Step S11.

In some embodiments, the method additionally comprises method steps S14, illustrated by the flow chart in FIG. 8B:
Step S14: receiving information about brain hemodynamics, electrical activity and impedance, from the at least one group of optical components 64 and from the at least one electrode array 82, 87, 88. In some embodiments, the received information includes neuronal responses surveilled by fNIRS, and/or changes in a concentration of oxyhemoglobin and/or hemoglobin or changes in blood flow in the vicinity of an optical component and/or change in electrical impedance detected using EIT.

In some embodiments, the method additionally comprises method step S15, illustrated by the flow chart in FIG. 8C:
Step S15: performing a brain-mapping using the received information of Step S14. In some embodiments, the brain-mapping is based upon information from at least two of: stereo-electroencephalography (sEEG), EIT performed within a lower range of two non-overlapping frequency ranges, EIT performed within an upper range of the two non-overlapping frequency ranges, and fNIRS. In some embodiments, the brain-mapping is based upon information from at least three of stereo-electroencephalography (sEEG), EIT performed within a lower range of two non-overlapping frequency ranges, EIT performed within an upper range of the two non-overlapping frequency ranges, and fNIRS. In some embodiments, the brain-mapping is based upon information from all four of: stereo-electroencephalography (sEEG), EIT performed within a lower range of two non-overlapping frequency ranges, EIT performed within an upper range of the two non-overlapping frequency ranges, and fNIRS. The lower range of the two non-overlapping frequency ranges can be below an upper threshold of 500 Hz, or 1 kHz, or 1.5 kHz, or 2 kHz. A second range of the two non-overlapping frequency ranges can be above a lower threshold of 3 kHz, or 2 kHz, or 1 kHz. In some embodiments, the information from fNIRS includes fNIRS information relating to multiple frequencies.

In some embodiments, the method additionally comprises method step S16, illustrated by the flow chart in FIG. 8D:
Step S16: positioning a thermal sensor 90 at least partly within a channel 95 of the plurality of channels 75, i.e., channel 95 can be a channel 75 also designated for passage therethrough of an optical emitter 63.

In some embodiments, the method additionally comprises method step S17, illustrated by the flow chart in FIG. 8E:
Step S17: receiving information about a thermal state, e.g., temperature, of the emitter 63 from a thermal, e.g., temperature, sensor 90.

In some embodiments, the method additionally comprises method steps S18, S19, which are illustrated by the flow chart in FIG. 8E. Method steps S18, S19 relate to a system 500 comprising multiple apparatuses 100 such that the intraosseous appliance 50 of steps S11-S16 is a first intraosseous appliance 50₁.

Step S18 inserting, in a 2nd cranial burr hole 215₂, a second intraosseous appliance 50₂ comprising a plurality of channels 75, 85 having respective proximal openings in a proximal extradermal surface 56.

Step S19 positioning at least one group of optical components 63, 64 at least partly within respective channels having distal openings in a distal base 59 of the second intraosseous appliance 50.

According to method steps S18, S19, an intracerebral optical component 64_{D} of the second apparatus 50 is configured to detect an emission by an intracerebral optical component 63_{D} of the first apparatus 50₁.

In some embodiments, the method is carried out so as to perform a monitoring of the brain. In some embodiments, the method is carried out so as to obtain a localization of one or more epileptogenic foci. In some embodiments, the method is carried out so as to obtain a characterization of neuro-vascular coupling.

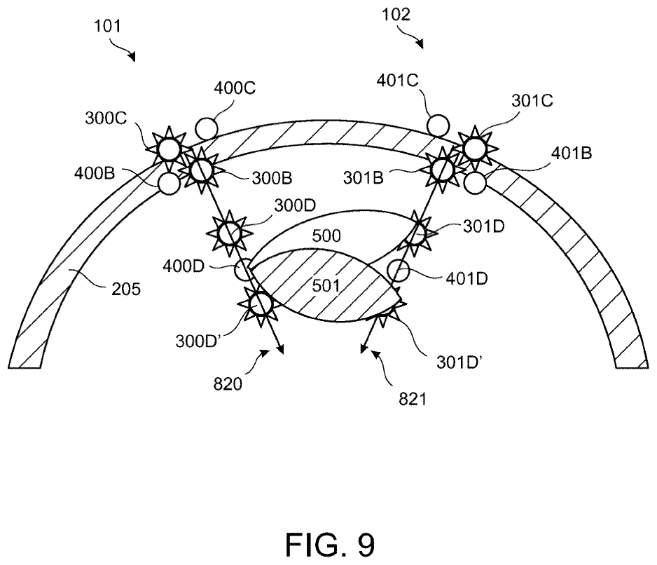

FIG. 9 illustrates embodiments of a system for brain imaging according to embodiments of the present disclosure.

Generally, a system for brain imaging according to the present disclosure may comprise one or more apparatuses (i.e. an intraosseous appliance and at least one optode as described above), the system including at least one emitter and at least one detector configured for example for functional near infrared spectroscopy (fNIRS). fNIRS enables to detect changes in hemoglobin concentration in a zone where the NIR signal is propagated from the emitter to the detector. An electronic circuitry of the system may enable to control light emission and reception and interpret the received signals.

The system for brain imaging may comprise a plurality of apparatuses as described in the present disclosure. In some of these embodiments, at least one detector of one apparatus may be configured to detect a signal emitted by at least one emitter on another apparatus.

FIG. 9 shows a system for brain imaging comprising two or more apparatuses 101, 102 according to the above in which an intracerebral detector 400D of one apparatus 101 is configured to detect a signal emitted by intracerebral emitters 301D, 301D′ of the other apparatus.

The apparatuses respectively include an intraosseous appliance (not shown to simplify the drawing) configured for reversible insertion in a burr hole of a patient skull 205 and having a hollow channel array that includes a plurality of channels. Optionally, the channels may have respective proximal openings in a proximal extradermal surface of the intraosseous appliance.

The hollow channel array comprises a second array subset comprising two or more channels adapted for respective passage therethrough of at least one emitter element 300B, 301B and at least one detector element 400B, 401B. The channels of the second array subset having respective distal openings in a lateral surface of the intraosseous appliance. In operation, the emitter elements 300B, 301B and the detector elements 400B, 401B are placed intraosseously so as to provide direct optical access to a lateral skull wall of the patient burr hole.

The hollow channel may alternatively or additionally comprise a first array subset comprising a plurality of channels adapted for respective passage therethrough of a first group of optical components including at least one emitter element and at least one detector element, the channels of the first array subset having respective distal openings in a distal base of the intraosseous appliance.

The hollow channel array comprises a third array subset comprising a plurality of channels adapted for respective passage therethrough of at least one emitter element 300C, 301C and at least one detector element 400C, 401C, the channels of the third array subset having respective distal openings in an extradermal surface of the intraosseous appliance that is not the proximal extradermal surface i.e. so as to provide direct optical access to a skin of a scalp portion peripheral to the patient burr hole. In operation, the emitters 300C, 301C and the detectors 400C, 401C are placed over the skin or scalp.

The hollow channel array may additionally comprise an electrode array including at least one electrode subarray including a first electrode subarray comprising one or more intracerebral (depth) electrodes 820, 821 adapted to reside at least partly within respective channels having distal openings in a distal base of the intraosseous appliance. The intracerebral electrodes 820, 821 respectively include at least a first and second optical emitters 300D, 301D, 300D′, 301D′ and at least one optical detector 400D, 401D configured for example for fNIRS. The emitters and/or detectors may be embedded on the depth electrodes. The optical emitters 300D, 300D′ and 301D, 301D′ are respectively placed at different longitudinal positions on the electrodes 820, 821 so as to reach different brain areas in operation. Several optical emitters may also be positioned at the same longitudinal position along the electrode. The optical emitters on any of the intracerebral electrodes may be emitting with different directions of illumination. For example, the emitters 300D, 300D′ may be optical fibers with side emitting apertures, such that light exiting these emitters is directed at different angles relative to a longitudinal axis of the electrode. Similarly, it is understood that more than one intracerebral detector may be positioned on the intracerebral electrode at different positions or at the same longitudinal position. An aperture of each detector determines the amount of light that can be collected by each detector. For example, a detector 400C collects light emitted from emitters 300D and 301D and should therefore have a large aperture (e.g. in the order of about 0.4 mm for example between 0.1 to 1.25 mm). An intracerebral detector inserted into the brain tissue may have a more limited aperture to allow flexibility and reduce potential damage to the tissue (e.g. in the order of about 200 microns, for example between 10 to 300 microns). The selection of different apertures for different detectors may allow for a flexible design of the imaging system.

It is noteworthy, that in some alternative embodiments, the optical emitters and detectors may not be mounted on the intracerebral electrodes 820, 821 but directly inserted in the first array subset described herein above. In these embodiments, the electrode array may be omitted.

In operation, the light exiting all emitters 300B, 300C, 300D, 300D′ and 301B, 301C, 301D, 301D′ is highly scattered by the tissue. Additionally, when either the detector or the emitter is placed inside the tissue, the light beam from the emitter reaching the detector does not follow a standard banana-like shape (i.e. curved and oblong) as is the case when both emitters and detectors are placed outside the tissue and light can escape from the system without being re-scattered. Propagation volumes 500, 501 represent a typical onion-shaped (i.e. ovoid) distribution of the light beam respectively from intracerebral emitters 301C, 301D to intracerebral detector 400D. Such a light distribution may be observed for every pair of emitter and detector when at least one of the emitter or detector is placed inside the tissue or bone and the distance of such element from the skin is higher than about 10 mean-free paths of light in the tissue. Only two such volumes are drawn for the sake of clarity.

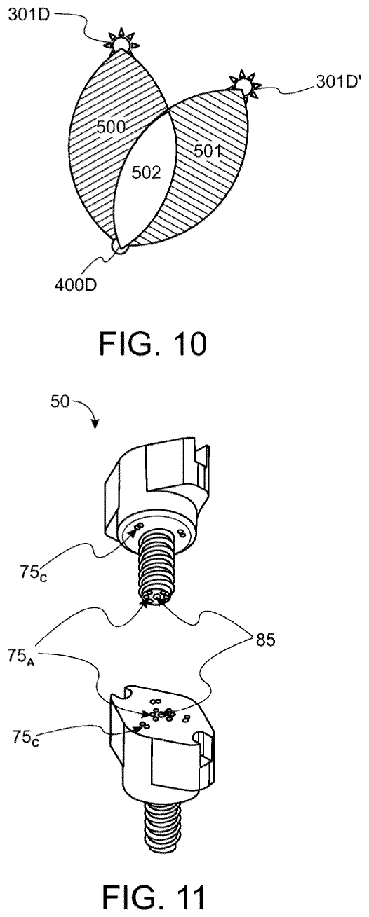

FIG. 10 shows an overlapping propagation volume 502 between propagation volumes 500 and 501 from emitters 301D and 301D′ on electrode 821 reaching detector 400D on intracerebral electrode 820 when the emitters operate using continuous wave illumination. It is understood that a system configuration including an overlapping propagation volume enables improving a spatial resolution for detection of hemodynamic changes. This is because when a change in light propagation properties (e.g. absorption or scattering due to hemodynamic changes) occurs within the overlapping volume 502, it results in a change of the detected signals by detector 400D resulting from light emitted by emitters 301D and 301D′ (either separately or simultaneously). Consequently, a model for the propagation of incoherent light between the two emitters 301D, 301D′ and detector 400D can enable detecting whether a change of the detected signal at detector 400D results from propagation through the overlapping volume 502 or outside of said overlapping volume. It is further understood that this method is not limited to the two emitters and one detector provided hereinabove. The higher the number of optodes, the better the spatial resolution improvement is.

Therefore, the present disclosure also provides a system including a plurality of intraosseous appliances. Each intraosseous appliance comprises at least one optode channel extending through the intraosseous appliance, said optode channel being configured to accommodate an optode and to provide direct optical access beyond a scalp of said patient. The at least one optode channel comprises one or more brain optode channel configured to provide direct optical access to a dura of the patient and configured for accommodating an optode in the form of an optical fiber or a fiber bundle. The system is provided with optodes in the optode channels of the intraosseous appliances. At least one optode accommodated in one intraosseous appliance is configured to form a plurality of intracerebral emitters and at least another one optode accommodated in another one intraosseous appliance is configured to form a detector detecting signal from at least some of said plurality of intracerebral emitters.

A system according to embodiments of the present disclosure may enable that:
1. The contamination of the extracerebral signal detected by non-invasive optodes, can be reduced when one or more emitters and detectors are placed inside the bone and the tissue because propagation through the scalp may be prevented.
2. Small diameter optical fiber (which are more flexible) can be used as intracerebral optodes configured for light emission because the diameter of the emitting fibers does not affect the quality of the emitted optical signals. Consequently, emitters 300D or 300D′ can be an output of a small diameter optical fiber.
3. For the detection elements, since larger apertures provide a higher signal and a better SNR, larger elements should preferably be placed outside the organ, either within the bone (as skull wall detector 400B in the skull wall optode channel) or outside the skin (as extradermal detector 400C in the extradermal optode channel), and smaller elements and thinner fibers should be placed intra-parenchymally, to reduce mechanical damage to the tissue. It is noteworthy that when using non-invasive illumination and detection, a higher illumination energy is needed compared to intra-tissue illumination and detection, since a large fraction of the light escapes the tissue, and can no longer be used for interrogation of the tissue. Conversely, when at least one of the optodes is positioned inside the tissue, the light does not escape before detection, and can eventually be detected (for example direct light from emitter to detector) and therefore less energy is needed for a similar SNR.

The presently disclosed subject matter has been described using detailed descriptions of embodiments thereof that are provided by way of example and are not intended to limit the scope of the presently disclosed subject matter. The described embodiments comprise different features, not all of which are required in all embodiments of the presently disclosed subject matter. Some embodiments of the presently disclosed subject matter utilize only some of the features or possible combinations of the features. Variations of embodiments of the presently disclosed subject matter that are described and embodiments of the presently disclosed subject matter comprising different combinations of features noted in the described embodiments will occur to persons skilled in the art to which the presently disclosed subject matter pertains. Any of the features described herein with respect to the various apparatuses and their respective components can be combined to make new combinations not specifically disclosed herein for purposes of conciseness, and such combinations are well within the scope of the presently disclosed subject matter.

1-109. (canceled)

110. An intraosseous appliance configured for insertion in a burr hole of a patient skull, the intraosseous appliance comprising:
at least one optode channel extending through the intraosseous appliance;
wherein said optode channel is configured to accommodate an optode and to provide direct optical access beyond a scalp of said patient.

111. The intraosseous appliance of claim 110, wherein the at least one optode channel comprises one or more brain optode channel configured to provide direct optical access to a dura of the patient.

112. The intraosseous appliance of claim 110, wherein the at least one optode channel comprises one or more skull wall optode channel configured to provide direct optical access to a lateral skull wall of the patient burr hole.

113. The intraosseous appliance of claim 110, further comprising at least one extradermal optode channel extending through an extradermal portion of the intraosseous appliance, said extradermal optode channel being configured to accommodate an extradermal optode and to provide direct optical access to a skin of a scalp portion peripheral to the patient burr hole.

114. The intraosseous appliance of claim 110, further comprising a depth electrode channel extending through the intraosseous appliance, said depth electrode channel being configured to accommodate a depth electrode and to provide direct access to a dura of the patient.

115. The intraosseous appliance of claim 110, further comprising a surface electrode channel being configured to accommodate a surface electrode and to provide access to a scalp surface.

116. The intraosseous appliance of claim 110, further comprising a stabilization structure configured for securing the intraosseous appliance in the cranial burr hole.

117. The intraosseous appliance of claim 116, wherein the stabilization structure includes a threading on at least part of a lateral surface of the intraosseous appliance.

118. The intraosseous appliance of claim 110, further comprising a distal portion formed from a material that is substantially transparent to a least a portion of a 400 nm-2 μ section of an electromagnetic spectrum.

119. The intraosseous appliance of claim 110, further comprising an extradermal annular flange portion.

120. The intraosseous appliance of claim 119, further comprising at least one extradermal optode channel extending through an extradermal portion of the intraosseous appliance, said extradermal optode channel being configured to accommodate an extradermal optode and to provide direct optical access to a skin of a scalp portion peripheral to the patient burr hole and wherein the extradermal optode channel extends through the extradermal annular flange portion.

121. The intraosseous appliance of claim 110, wherein the at least one optode channel is included in a hollow channel array that includes a plurality of channels having respective proximal openings in a proximal extradermal surface of the intraosseous appliance, the hollow channel array comprising at least one array subset selected from:
a first array subset comprising a plurality of channels adapted for respective passage therethrough of a first group of optical components including at least one emitter element and at least one detector element, and a first electrode array comprising at least one electrode, the channels of the first array subset having respective distal openings in a distal base of the intraosseous appliance; or
a second array subset comprising a plurality of channels adapted for respective passage therethrough of a second group of optical components including at least one emitter element and at least one detector element, the channels of the second array subset having respective distal openings in a lateral surface of the intraosseous appliance.

122. The intraosseous appliance of claim 121, further comprising a third array subset comprising a plurality of channels adapted for respective passage therethrough of a third group of optical components including at least one emitter element and at least one detector element, and a second electrode array comprising at least one electrode, the channels of the third array subset having respective distal openings in an extradermal surface of the intraosseous appliance that is not the proximal extradermal surface.

123. An apparatus, comprising:
an intraosseous appliance shaped for insertion in, and removal from, a cranial burr hole, the intraosseous appliance comprising a plurality of channels having respective proximal openings in a proximal extradermal surface of the intraosseous appliance; and
at least one group of optical components, each of the optical components comprising one of an emitter and a detector, the at least one group selected from: (i) a first group of optical components adapted to reside at least partly within respective channels having distal openings in a distal base of the intraosseous appliance so as to establish respective optical paths between respective distal ends of the first-group optical components and a dura of the subject, or (ii) a second group of optical components adapted to reside at least partly within respective channels having distal openings in a lateral surface of the intraosseous appliance so as to establish respective optical paths between respective distal ends of the second-group optical components and a bone wall of the burr hole, and (iii) a third group of optical components adapted to reside at least partly within respective channels having distal openings in an extradermal surface of the intraosseous appliance that is not the proximal extradermal surface so as to establish respective optical paths between respective distal ends of the third-group optical components and skin of the subject.

124. The apparatus of claim 123, further comprising an electrode array including at least one electrode subarray selected from: (i) a first electrode subarray comprising one or more intracerebral electrodes adapted to reside at least partly within respective channels having distal openings in a distal base of the intraosseous appliance, or (ii) a second electrode subarray comprising one or more extracerebral electrodes adapted to reside at least partly within respective channels having distal openings in the extradermal surface of the intraosseous appliance that is not the proximal extradermal surface.

125. The apparatus of claim 123, wherein the apparatus comprises at least two groups of optical components selected from the first, second and third groups of optical components.

126. The apparatus of claim 123, wherein the apparatus comprises the first, second and third groups of optical components.

127. The apparatus of claim 123, further comprising one or more intracerebral optical components configured to detect or emit near infrared light.

128. The apparatus of claim 123, wherein at least some of the optical components are configured for functional near infrared spectroscopy (fNIRS) surveillance of hemodynamic responses and/or for fNIRS surveillance of neuronal responses.

129. The apparatus of claim 123, wherein at least some of the optical components are configured for measuring a change a concentration of oxyhemoglobin and/or hemoglobin in the vicinity of the at least some of the optical components.

130. The apparatus of claim 123, wherein at least some of the electrodes of the electrode array are configured for detecting and imaging electrical impedance change using electrical impedance tomography (EIT).
